(19) United States
(12) Patent Application Publication
(10) Pub. No.: US 2025/0281143 A1
(43) Pub. Date: Sep. 11, 2025

(54) METHODS AND SYSTEMS FOR SETTING PROTOCOL PARAMETERS

(71) Applicant: SHANGHAI UNITED IMAGING HEALTHCARE CO., LTD., Shanghai (CN)

(72) Inventors: Yang XIN, Shanghai (CN); Guobin LI, Shanghai (CN); Zhenkui WANG, Shanghai (CN); Li TONG, Shanghai (CN); Zhongqi ZHANG, Shanghai (CN); Xiaomao GONG, Shanghai (CN)

(73) Assignee: SHANGHAI UNITED IMAGING HEALTHCARE CO., LTD., Shanghai (CN)

(21) Appl. No.: 19/074,502

(22) Filed: Mar. 10, 2025

(30) Foreign Application Priority Data
Mar. 8, 2024 (CN) 202410267244.X
Aug. 30, 2024 (CN) 202411216272.5

(51) Int. Cl. A61B 6/00; A61B 6/46

(52) U.S. Cl. CPC A61B 6/542; A61B 6/465

(57) ABSTRACT

A method for setting protocol parameters, including: obtaining a current setting value of a target protocol parameter in a scanning protocol to be verified, the current setting value being set by a user through a terminal device; determining a statistical distribution situation of historical setting values of the target protocol parameter and a relative position of the current setting value in the statistical distribution situation; and generating prompt information relating to parameter setting based on the relative position.

### CROSS-REFERENCE TO RELATED APPLICATION

This claims priority of Chinese Patent Application No. 202410267244.X, filed on Mar. 8, 2024, and the Chinese Patent Application No. 202411216272.5, filed on Aug. 30, 2024, the contents of each of which are entirely incorporated herein by reference.

### TECHNICAL FIELD

The present disclosure relates to the field of medical imaging technology, and in particular relates to methods and systems for setting protocol parameters.

### BACKGROUND

Medical imaging devices are often used to scan a patient and obtain a medical image of the patient to assist in treatment or diagnosis. Common medical imaging devices include a positron emission tomography (PET), a magnetic resonance imaging (MRI) device, etc. Before the scan, it is usually necessary for a user (e.g., a physician, a medical technician, etc.) to select a scanning protocol in a protocol configuration interface of the medical imaging device and set or adjust a parameter of each protocol parameter in the scanning protocol. After the scan is completed and a scanning image is obtained, the user determines if the protocol parameter is set appropriately. If the user determines that the protocol parameter is not set appropriately, the user needs to modify the protocol parameter to rescan the patient until the obtained scanning image satisfies the requirement. This trial-and-error process of adjusting the protocol parameter leads to a longer scanning time and a lower scanning efficiency and accuracy.

Therefore, it is desirable to provide methods and systems for setting protocol parameters efficiently and accurately.

### SUMMARY

Embodiments of the present disclosure provide a method for setting protocol parameters including: obtaining a current setting value of a target protocol parameter in a scanning protocol to be verified, the current setting value being set by a user through a terminal device; determining a statistical distribution situation of historical setting values of the target protocol parameter and a relative position of the current setting value in the statistical distribution situation; and generating prompt information relating to parameter setting based on the relative position.

Embodiments of the present disclosure provide a system for setting protocol parameters including a processing device and a display device. The display device is configured to display a current setting value of a target protocol parameter on a graphical user interface, the processing device is configured to obtain the current setting value, perform a reasonableness verification on the current setting value to generate a parameter verification result, and determine prompt information relating to parameter setting based on the parameter verification result, and the display device is further configured to display the prompt information on the graphical user interface, the prompt information indicating that the current setting value of the target protocol parameter needs to be adjusted.

Embodiments of the present disclosure provide a system for setting protocol parameters, including: at least one storage device storing a set of instructions; and at least one processor configured to communicate with the at least one storage device. When executing the set of instructions, the at least one processor is configured to direct the system to perform operations including: in response to user operations on a graphical user interface of a terminal device, selecting a scanning protocol and a current setting value of a target protocol parameter of the scanning protocol; obtaining and displaying a parameter value distribution chart corresponding to the scanning protocol on the graphical user interface, the parameter value distribution chart having an annotation that indicates a relative position of the current setting value in the parameter value distribution chart; and generating a target scanning protocol in response to user adjustment instructions on the graphical user interface with respect to the current setting value.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure will be further illustrated by way of exemplary embodiments, which are described in detail by means of the accompanying drawings. These embodiments are non-limiting exemplary embodiments, in which like reference numerals represent similar structures throughout the several views of the drawings, and wherein:

FIG. 1 is a schematic diagram illustrating an application scenario of a system for setting protocol parameters according to some embodiments of the present disclosure;

FIG. 2 is a flowchart illustrating an exemplary method for setting protocol parameters according to some embodiments of the present disclosure;

FIG. 3 is a schematic diagram illustrating a graphical user interface according to some embodiments of the present disclosure;

FIGS. 4A-4D are schematic diagrams illustrating parameter value distribution charts according to some embodiments of the present disclosure;

FIG. 5 is a schematic diagram illustrating a parameter value distribution chart according to some embodiments of the present disclosure;

FIG. 6 is a schematic diagram illustrating a parameter value distribution chart according to some embodiments of the present disclosure;

FIG. 7 is a schematic diagram illustrating a parameter value distribution chart according to some embodiments of the present disclosure;

FIG. 8 is a schematic diagram illustrating a graphical user interface according to some embodiments of the present disclosure;

FIG. 9 is a schematic diagram illustrating an abnormity alert according to some embodiments of the present disclosure;

FIG. 10 is a flowchart illustrating an exemplary process for determining a scanning protocol to be verified according to some embodiments of the present disclosure;

FIG. 11 is a schematic diagram illustrating a pop-up displaying a scanning protocol to be verified according to some embodiments of the present disclosure;

FIG. 12 is a schematic diagram illustrating a graphical user interface according to some embodiments of the present disclosure;

FIG. 13 is a schematic diagram illustrating a system for setting protocol parameters according to some embodiments of the present disclosure;

FIG. 14 is a schematic diagram illustrating a computer device according to some embodiments of the present disclosure;

FIG. 15 is a flowchart illustrating an exemplary process for generating a target scanning protocol according to some embodiments of the present disclosure;

FIG. 16 is a flowchart illustrating an exemplary process for updating a protocol database according to some embodiments of the present disclosure;

FIG. 17 is a schematic diagram illustrating a process for updating a protocol database according to some embodiments of the present disclosure;

FIG. 18 is a flowchart illustrating an exemplary process for constructing a protocol database according to some embodiments of the present disclosure;

FIG. 19 is a flowchart illustrating an exemplary process for setting protocol parameters according to some embodiments of the present disclosure;

FIG. 20 is a flowchart illustrating an exemplary process for determining a target scanning protocol according to some embodiments of the present disclosure;

FIG. 21 is a flowchart illustrating an exemplary process for determining a target scanning protocol according to some embodiments of the present disclosure; and

FIG. 22 is a flowchart illustrating an exemplary method for setting protocol parameters according to some embodiments of the present disclosure.

### DETAILED DESCRIPTION

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

The terminology used herein is for the purpose of describing particular example embodiments only and is not intended to be limiting. As used herein, the singular forms “a,” “an,” and “the” may be intended to include the plural forms as well, unless the context clearly indicates otherwise. It will be further understood that the terms “comprise,” “comprises,” and/or “comprising,” “include,” “includes,” and/or “including,” when used in this specification, specify the presence of stated features, integers, steps, operations, elements, and/or components, but do not preclude the presence or addition of one or more other features, integers, steps, operations, elements, components, and/or groups thereof.

It will be understood that when a unit, engine, module, or block is referred to as being “on,” “connected to,” or “coupled to,” another unit, engine, module, or block, it may be directly on, connected or coupled to, or communicate with the other unit, engine, module, or block, or an intervening unit, engine, module, or block may be present, unless the context clearly indicates otherwise. As used herein, the term “and/or” includes any and all combinations of one or more of the associated listed items.

Flowcharts are used in the present disclosure to illustrate operations performed by a system in accordance with embodiments of the present disclosure. It should be appreciated that the preceding or following operations are not necessarily performed in an exact sequence. Instead, operations may be processed in reverse sequence or simultaneously. Also, it is possible to add other operations to these processes, or to remove an operation or operations from these processes.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure. It is understood that the drawings are not to scale.

Protocol parameters in a scanning protocol need to be set before scanning and imaging. Taking magnetic resonance imaging (MRI) as an example, it enables a non-invasive and radiation-free structural and functional imaging, which is a key technology for clinical detecting and targeting a variety of diseases. However, setting parameters of an MRI sequence is a complex task, which is quite challenging for average users. First, MRI involves a wide variety of sequences that are continually evolving, each with its unique technical features, image performances, and clinical scenarios, which require a great deal of time and effort for an operator to familiarize with and understand. Second, MRI involves a great count of protocol parameters, which have interactions and dependencies with each other, and optimization and adjustment of the protocol parameters are complicated. To achieve an ideal combination of protocol parameters, the operator needs to consider an organizational feature and an application scenario, and also a performance feature of the scanning device itself.

Currently, the operator needs to set and adjust the protocol parameter in the scanning protocol. However, the experience and technical skill levels of the operator become one of the key factors that affect the scanning effectiveness. A stable MRI scanning requires operators with in-depth theoretical knowledge and extensive practical experiences to accurately identify and solve problems. Inexperienced operators may misinterpret or incorrectly set the protocol parameters, resulting in a poor scanning quality. In daily scanning, even experienced technicians only master the adjustment of an individual simple protocol parameter, such as a view angle, a layer count, a layer thickness, a layer spacing, a saturated band, a flip angle, etc. The adjustment of the protocol parameters for complex targets is heavily dependent on the training and technical support of device manufacturers.

It should be understood that the above example of MRI is for exemplary purposes only, and that the technical solutions disclosed in some embodiments of the present disclosure may apply to other imaging techniques that require the setting of protocol parameters.

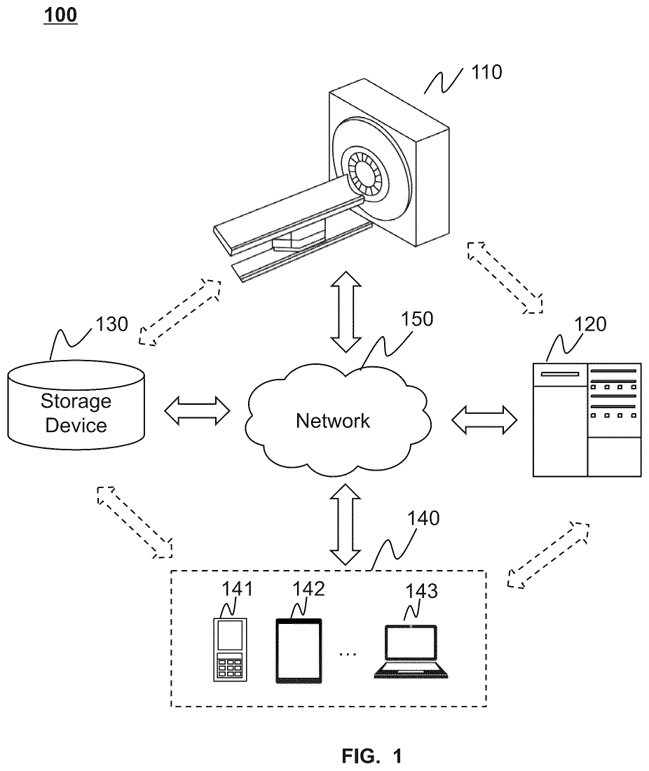

FIG. 1 is a schematic diagram illustrating an application scenario of a system for setting protocol parameters according to some embodiments of the present disclosure.

As shown in FIG. 1, a system 100 (referred to as system 100 for brevity) includes a scanning device 110, a processing device 120, a storage device 130, a terminal device 140, and a network 150. In some embodiments, at least two components of the system 100 are connected to and/or communicate with each other via a wireless connection, a wired connection, or a combination thereof. The system 100 may include various types of connections between its components. For example, the scanning device 110 is connected to the processing device 120 through the network 150, or directly connected to the processing device 120 as shown by the bidirectional dashed arrow connecting the scanning device 110 and the processing device 120 in FIG. 1.

The scanning device 110 may be configured to collect imaging data associated with a scanning object. The imaging data associated with the scanning object may include an image (e.g., an image slice), projection data, or a combination thereof. In some embodiments, the imaging data includes two-dimensional (2D) imaging data, three-dimensional (3D) imaging data, four-dimensional (4D) imaging data, etc. or any combination thereof. The scanning object may be biological or non-biological. For example, the scanning object includes a patient, an artificial object, etc. For another example, the scanning object may include a particular portion, organ, and/or tissue of a patient. For example, the scanning object includes a head, a neck, a chest, a heart, a stomach, a blood vessel, a soft tissue, a tumor, etc., or any combination thereof.

In some embodiments, the scanning device 110 includes a single modality imaging device. For example, the scanning device 110 includes a positron emission tomography (PET) device, a single photon emission computed tomography (SPECT) device, an MRI device (also referred to as an MR device, an MR scanner), a computed tomography (CT) device (e.g., a helical CT, an e-beam CT, an energy-spectrum CT), an ultrasound (US) device, an X-ray imaging device, a digital subtraction angiography (DSA) device, a magnetic resonance angiography (MRA) device, a computed tomography angiography (CTA) device, etc., or any combination thereof. In some embodiments, the scanning device 110 includes a multi-modality imaging device. Exemplary multi-modality imaging devices include a PET-CT device, a PET-MRI device, a SPET-CT device, etc., or any combination thereof. The multi-modality imaging device may perform multi-modality imaging simultaneously. For example, the PET-CT device collects both structural X-ray CT data and functional PET data in a single scanning. The PET-MRI device collects both MRI data and PET data in a single scanning.

In some embodiments, the processing device 120 determines a setting value of a protocol parameter of the scanning device 110, and the scanning device 110 scans the scanning object based on the determined setting value of the protocol parameter to obtain a medical image.

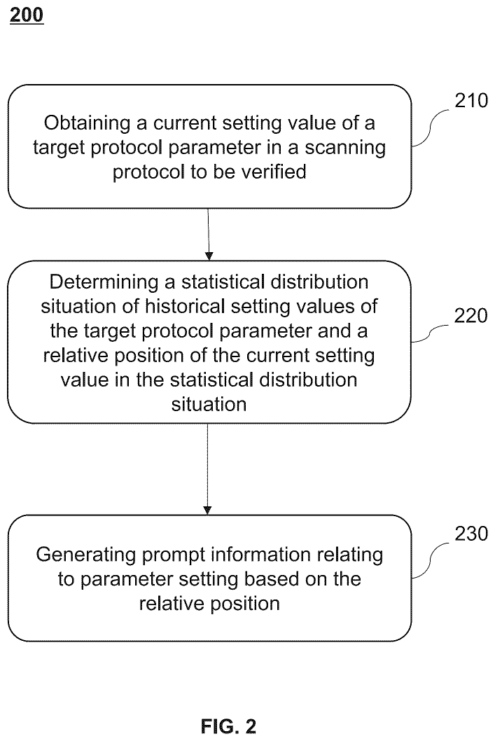

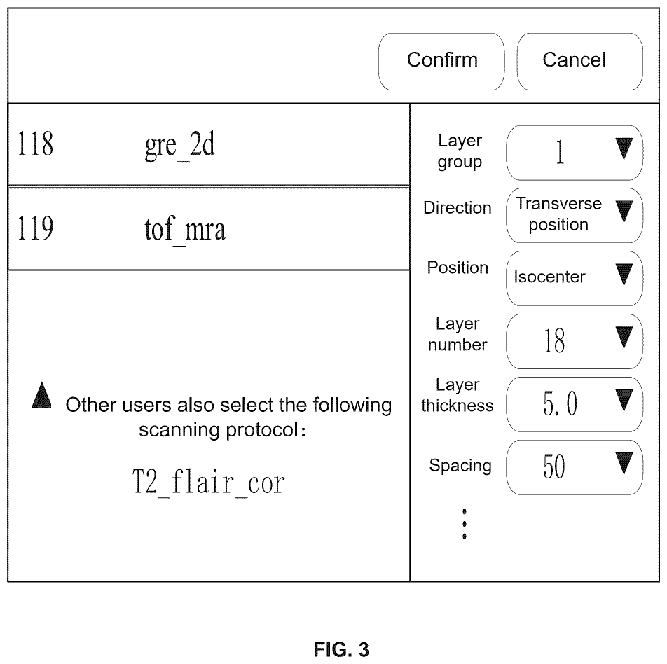

The processing device 120 may process data and/or information. The data and/or information may be obtained from the scanning device 110 or retrieved through the network 150 from the storage device 130, the terminal device 140, and/or an external device (external to the system 100). In some embodiments, the processing device 120 obtains a current setting value of a target protocol parameter in a scanning protocol to be verified, the current setting value being set by a user through a terminal device; determines a statistical distribution situation of historical setting values of the target protocol parameter and a relative position of the current setting value in the statistical distribution situation; and generates prompt information relating to parameter setting based on the relative position. More descriptions of the above embodiment may be found elsewhere in the present disclosure (e.g., the relevant descriptions of FIGS. 2 and 3, etc.).

In some embodiments, the processing device 120 includes an obtaining module, a determination module, and a prompting module. The obtaining module is configured to obtain the current setting value of the target protocol parameter in the scanning protocol to be verified. More descriptions about obtaining the current setting value may be found in the descriptions of operation 210.

The determination module is configured to determine the statistical distribution situation of historical setting values of the target protocol parameter and the relative position of the current setting value in the statistical distribution situation. More descriptions about determining the statistical distribution situation and the relative position may be found in the descriptions of operation 220.

The prompting module is configured to generate the prompt information relating to parameter setting based on the relative position. More descriptions about generating the prompt information may be found in the descriptions of operation 230.

In some embodiments, the processing device 120 is a single server or a server group. The server group is centralized or distributed. In some embodiments, the processing device 120 is local or remote. In some embodiments, the processing device 120 is implemented on a cloud platform. For example, the cloud platform includes a private cloud, a public cloud, a hybrid cloud, a community cloud, a distributed cloud, an interconnected cloud, a diverse cloud, etc., or any combination thereof. In some embodiments, the processing device 120 is a part of the scanning device 110 or the terminal device 140.

The storage device 130 stores data, instructions, and/or any other information. In some embodiments, the storage device 130 stores data obtained from the scanning device 110, the processing device 120, and/or the terminal device 140. The data includes historical scanning protocols, a protocol database, etc. For example, the storage device 130 stores historical scanning protocols used in historical scanning processes, etc.

In some embodiments, the storage device 130 stores data and/or instructions that the processing device 120 and/or the terminal device 140 executes or uses to perform exemplary methods described in the present disclosure. In some embodiments, the storage device 130 includes a mass memory, a removable memory, a volatile read-write memory, a read-only memory (ROM), etc., or any combination thereof. In some embodiments, the storage device 130 is implemented on the cloud platform. Merely by way of example, the cloud platform includes the private cloud, the public cloud, the hybrid cloud, the community cloud, the distributed cloud, the interconnected cloud, the diverse cloud, etc., or any combination thereof. In some embodiments, the storage device 130 is integrated into the scanning device 110 or the terminal device 140.

The terminal device 140 is connected to and/or in communication with the scanning device 110, the processing device 120, and/or the storage device 130. In some embodiments, the terminal device 140 includes a mobile device 141, a tablet computer 142, a laptop computer 143, etc., or any combination thereof. In some embodiments, the terminal device 130 is integrated into the scanning device 110.

In some embodiments, the system 100 further includes a display device. The display device displays information (e.g., the current setting value of the target protocol parameter, a parameter value distribution chart, the prompting information, etc.) to the user through a graphical user interface, and receives information and instructions input by the user. In some embodiments, the display device is integrated into the terminal device 140. More descriptions of the display device may be found in FIG. 13, etc.

The network 150 may include any suitable network capable of facilitating an exchange of information and/or data for the system 100. In some embodiments, at least one component of the system 100 (e.g., the scanning device 110, the processing device 120, the storage device 130, the terminal device 140, etc.) transmits information and/or data with at least one other component of the system 100 through the network 150. In some embodiments, the network 150 includes at least one network access point. For example, the network 150 includes a wired and/or wireless network access point, such as a base station and/or an Internet exchange point, through which at least one of the components of the system 100 connects to the network 150 to exchange the data and/or information.

The descriptions of the system 100 are intended to be illustrative and not to limit the scope thereof. Numerous substitutions, modifications, and variations are apparent to those skilled in the art. The features, structures, methods, and other characteristics of the exemplary embodiments described in the present disclosure are combined in various ways to obtain additional and/or alternative exemplary embodiments. These variations and modifications, however, do not depart from the scope of the present disclosure. In some embodiments, the system 100 includes at least one additional component and/or at least one component of the system 100 described above is omitted. Additionally, the at least two components of the system 100 are integrated into a single component. The components of the system 100 may be implemented on at least two sub-components.

FIG. 2 is a flowchart illustrating an exemplary method for setting protocol parameters according to some embodiments of the present disclosure. In some embodiments, a process 200 is performed by a processor (e.g., the processing device 120, etc.). As shown in FIG. 2, the process 200 includes the following operations.

In 210, a current setting value of a target protocol parameter in a scanning protocol to be verified may be obtained. In some embodiments, the current setting value is set by a user through a terminal device. The user may be a medical technologist, an operator of the scanning device 110, etc.

The scanning protocol to be verified refers to a scanning protocol whose reasonableness needs to be verified. The scanning protocol is used to describe a scanning manner and protocol parameters when scanning a scanning object. Taking the scanning protocol of an MR device as an example, the scanning protocol records an MR scanning type, a scanning sequence, a scanning position, etc. The MR scanning type is used to characterize a signal of liquid in an MR scanning image, and the MR scanning type includes but is not limited to, T1-weighted pulse sequence and T2-weighted pulse sequences. A T1-weighted pulse sequence is implemented to generate T1-weighted MR images of a desired region of the examination subject. A T2-weighted pulse sequence is implemented to generate T2-weighted MR images. The scanning sequence indicates a combination of pulses used, which includes a spin echo (SE) sequence, a fast spin echo (FSE) sequence, an inversion recovery (IR) sequence, and an echo planar (EP) sequence. The scanning position includes a transverse (TRA), an axial (AX), a sagittal (SAG), and a coronal (COR).

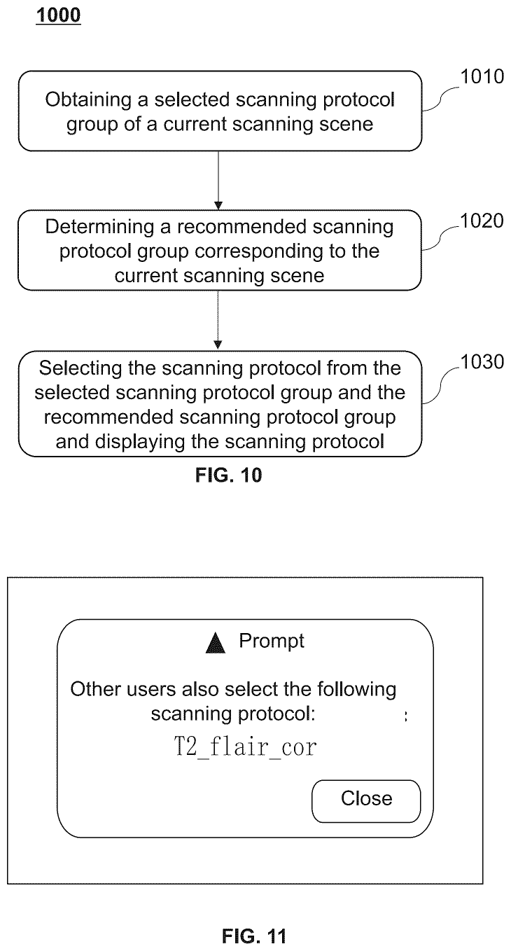

The scanning protocol to be verified may be selected by the user or determined by the processing device 120. For example, the processing device 120 determines the scanning protocol to be verified through the process 1000 shown in FIG. 10. For another example, the processing device 120 determines the scanning protocol corresponding to a target scan to be performed as the scanning protocol to be verified.

The target protocol parameter refers to a protocol parameter that needs to be verified in the scanning protocol. The protocol parameter refers to a parameter related to the scanning process. For example, in the case of MRI, the target protocol parameter includes a field of view (FOV), a layer thickness, a layer spacing, a time of repetition (TR), a number of excitation (NEX), etc. In some embodiments, the protocol parameter is also referred to as a scanning parameter. The target protocol parameter includes all or part of the protocol parameters in the scanning protocol.

The current setting value refers to the content of the target protocol parameter at a current moment (e.g., before verification). For example, the current setting value is that a layer group is 1, a direction is a transverse position, and a layer number is 18, etc. The current setting value includes a numeric setting value or an option setting value. For example, the setting value of protocol parameters such as the layer group, the layer number, a layer thickness, a spacing, etc., is the numeric setting value, which is a numerical value within a specific range; the setting value of protocol parameters such as the direction, a position, the type, etc. is the option setting value, which is selected from preset options. In some embodiments, optional numeric setting values may be provided to the user for selection to facilitate parameter setting.

FIG. 3 is a schematic diagram illustrating a graphical user interface according to some embodiments of the present disclosure. The graphical user interface is displayed to a user through the terminal device 140. As shown in FIG. 3, the left-side region on the graphical user interface is a protocol selection region, and the processing device reads that a scanning protocol selected by the user in the protocol selection region is tof_mra in FIG. 3, then takes tof_mra as a scanning protocol to be verified. Tof_mra is a time of flight magnetic resonance angiography (MRA) protocol, which is a key scanning protocol for head scanning and is usually used for the diagnosis of cerebral aneurysm, cerebral vascular stenosis, and vascular developmental abnormality, etc. The tof_mra has features of a long scanning time and a complex parameter combination.

Further, the user may set a current setting value of a target protocol parameter through a graphical user interface as shown in FIG. 3. Exemplarily, as shown in FIG. 3, the right-side region of the graphical user interface is a parameter setting region, which is used by the user to set a value of the protocol parameter. The “layer group, direction, position . . . ” are parameter names, “1, transverse slice, isocenter . . . ” are the setting values corresponding to the parameter names. Optionally, when the user performs the parameter setting operation, the processing device may obtain, in real time, the current setting value of a target protocol parameter set on the graphical user interface. Alternatively, the processing device may obtain the current setting value of the target protocol parameter set on the graphical user interface after the user triggers a confirmation instruction after parameter setting. For example, the user triggers the confirmation instruction by clicking “confirmation” in FIG. 3. It should be noted that each type of scanning protocol corresponds to a plurality of protocol parameters, and the current setting value of the target protocol parameter obtained as described above includes the parameter names and parameter contents of all or part of the protocol parameters of the scanning protocol.

In some embodiments, the user selects the scanning protocol (e.g., by inputting a label of the scanning protocol) through the graphical user interface, and the processing device 120 searches for the scanning protocol from a protocol database and displays the target protocol parameter corresponding to the scanning protocol and the current setting value of the target protocol parameter on the graphical user interface. At this time, the current setting value of the target protocol parameter is obtained by auto-matching after the scanning protocol is selected by the user. The current setting value obtained by the automatic matching may be the value set by a previous user or in a previous scanning, or may be pre-configured in the system. Descriptions of the label of the scanning protocol and the protocol database may be found in operation 220.

In 220, a statistical distribution situation of historical setting values of the target protocol parameter and a relative position of the current setting value in the statistical distribution situation may be determined.

The historical setting values of the target protocol parameter refer to setting values of the target protocol parameter in historical scanning protocols. The historical scanning protocols refer to scanning protocols used during historical scanning, for example, the historical scanning protocols include scanning protocols used in historical scanning in past time periods. For example, within the past one day, within the past 3 days, within the past 7 days, etc.

In some embodiments, the processing device 120 determines target historical scanning protocols corresponding to the scanning protocol; for each of the target historical scanning protocols, determines a quality assessment result of a scanning image corresponding to the target historical scanning protocol; selects reference scanning protocols whose quality assessment results satisfy preset requirements from the target historical scanning protocols; and determines the statistical distribution situation based on the historical setting values of the target protocol parameter in the reference scanning protocols.

The target historical scanning protocols refer to historical scanning protocols corresponding to the scanning protocol. For example, a target historical scanning protocol is a historical scanning protocol that has the same name as the scanning protocol, a historical scanning protocol that corresponds to the same scanning body part as the scanning protocol, etc. In some embodiments, the processing device 120 determines the corresponding target historical scanning protocols based on the name of the scanning protocol. Taking the tof_mra protocol shown in FIG. 3 as an example, the processing device 120 designates historical scanning protocols named tof_mra as the target historical scanning protocols.

The scanning image corresponding to a target historical scanning protocol refers to a historical scanning image obtained after the target historical scanning protocol is executed. The quality assessment result of the scanning image is used to characterize a quality level of the scanning image. Exemplarily, the quality assessment result includes assessment values corresponding to assessment indicators such as a clarity, a signal-to-noise ratio (SNR), a contrast ratio, a presence of artifacts, etc. As another example, the quality assessment result includes an overall image quality grade (e.g., a high quality, a medium quality, a low quality, etc.). In some embodiments, both the assessment values and the overall image quality grade are expressed in a form of a numerical value (score), e.g., a score of 1-10, with a greater score indicating a better quality of the corresponding scanning image.

In some embodiments, the quality assessment result of the scanning image is labeled by a user (e.g., an expert). In some embodiments, after obtaining the scanning image, the processing device 120 inputs the scanning image into an image quality assessment model, and obtains the quality assessment result output from the image quality assessment model. The image quality assessment model is a neural network model, e.g., a deep neural network model, obtained by training an initial model using sample images. The image quality assessment model is used for assessing the quality level of the input image. In some embodiments, the image quality assessment model is obtained by training an initial model based on a first sample and a first label. The first sample includes a sample scanning image, and the first label is a manually labeled image quality assessment result. The image quality assessment models may be trained by common model training methods (e.g., a gradient descent, etc.).

The preset requirements may include that the assessment values corresponding to one or more assessment indicators in the quality assessment result are within a preset range or that an overall quality grade is higher than or equal to a preset grade. For example, the clarity is a high definition; for another example, the quality assessment result is a high quality grade, etc. For another example, an overall score of the quality assessment result is greater than or equal to 7.

In some embodiments, the processing device 120 selects, based on the quality assessment results of the scanning images, one or more historical scanning protocols from the plurality of target historical scanning protocols whose quality assessment results satisfy the preset requirements as the reference scanning protocols. The reference scanning protocol refers to a scanning protocol selected from the target historical scanning protocols to be used as a reference in determining the statistical distribution situation.

A reference scanning protocol includes the setting value of each protocol parameter during a historical scanning. In some embodiments, the processing device 120, when determining the reference scanning protocols, also determines a number of uses of historical setting values of each protocol parameter included therein (i.e., how often the historical setting values of the protocol parameter are used by the user). The number of uses and the historical setting values are displayed in different manners (e.g. in different colors). By providing the number of uses, it helps the user to understand more about the selections of a user population in setting the protocol parameters, and provides the user with more reference information about the parameter setting.

After determining the reference scanning protocols, the processing device 120 determines the statistical distribution situation of the target protocol parameter based on the historical setting values of the target protocol parameter in the reference scanning protocol. The statistical distribution situation is used to reflect a numerical distribution situation of the historical setting values of the target protocol parameter. The numerical distribution situation is used to reflect a frequency, a pattern, a concentration trend, and a degree of dispersion of the various historical setting values of the target protocol parameter.

For example, the statistical distribution situation includes coordinate charts, bar charts, sector charts (also referred to as a dashboard chart), tables, statistical values (e.g., averages), etc., that are used to represent the distribution of the historical setting values. Taking a coordinate chart as an example, a horizontal axis indicates the historical setting value, and a vertical axis indicates a frequency of use of the historical setting value.

It should be understood that the above description of the process for determining the statistical distribution situation is for illustrative purposes only. In some other embodiments, the processing device 120 directly determines the statistical distribution situation based on the historical setting values of the target protocol parameter in the historical scanning protocols or the target historical scanning protocols.

In some embodiments, the statistical distribution situation includes a reference value range of the target protocol parameter. The reference value range of the target protocol parameter refers to a normal or typical range that the value of the target protocol parameter is in during historical scanning. Taking a numerical target protocol parameter as an example, the processing device determines upper and lower value limits based on the historical setting values, and uses a value range bounded by the upper and lower value limits as the reference value range. For example, for scanning protocol A that includes a numerical protocol parameter m, the scanning images that satisfy the quality assessment result include a1-a100, and each scanning image corresponds to a historical setting value of one protocol parameter m, the scanning images a1-a100 correspond to 100 historical setting values of the protocol parameter m. The processing device 120 determines the upper value limit m1 and the lower value limit m2 based on these 100 historical setting values of the protocol parameter m, respectively, to obtain a reference value range [m1, m2] of the protocol parameter m, and the reference value range is taken as the statistical distribution situation of the protocol parameter m. For an optional protocol parameter, the processing device may determine an option content that occurs more frequently than a threshold based on the historical setting values as the reference value range of the option content (also referred to as a reference option content). For example, for a scanning protocol B that includes an optional protocol parameter n, the scanning images that satisfy the quality requirements include b1-b100, and each scanning image corresponds to an option content, and the scanning images b1-b100 correspond to 100 option contents. The processing device determines a high-frequency option content based on these 100 option contents and takes the high-frequency option content as the reference option content of the protocol parameter n. Referring to FIG. 3, the setting values of the protocol parameters such as the layer group, the layer count, the layer thickness, the spacing, etc., in the figure are the numerical setting values, and the corresponding reference value ranges are numerical ranges, e.g., the reference value range for the layer thickness is 0.7-1.0. The setting values of the protocol parameters such as the direction, the position, and the type in the figure are optional setting values, and the corresponding reference value ranges are the optional contents, e.g., the reference value range of the position is the transverse position.

In some embodiments, the processing device 120 determines the reference value range of the target protocol parameter based on the protocol database and takes the reference value range as the statistical distribution situation. The protocol database refers to a database containing various scanning protocols, the historical setting values and/or the statistical results of the various scanning protocols, etc. For example, the protocol database stores reference value ranges of a plurality of protocol parameters of various types of reference scanning protocols. Each protocol parameter in a type of reference scanning protocol (e.g., the tof_mra protocol shown in FIG. 3) has a corresponding reference value range. In some embodiments, the protocol database is statistically obtained based on a great amount of sample data. The protocol database may be updated periodically or irregularly. The protocol database may be a local database or a remote database (e.g., a cloud database). In some embodiments, the protocol database is a database that is shared by a plurality of organizations jointly.

In some embodiments, the statistical distribution situation includes a parameter value distribution chart of the target protocol parameter. The parameter value distribution chart is a graph indicating a distribution of the historical setting values of the target protocol parameter. In some embodiments, the parameter value distribution chart shows how often each of the historical setting values is used. In some embodiments, the scanning protocol corresponds to a plurality of protocol parameters, each protocol parameter has a corresponding parameter value distribution chart, or only key parameters (parameters that with a great impact on the scanning and imaging) among the plurality of protocol parameters have corresponding parameter value distribution charts. In some embodiments, the parameter value distribution charts are pre-stored within the storage device 150 or stored in the cloud. For example, the parameter value distribution charts are obtained from the protocol database.

In some embodiments, the parameter value distribution chart includes one or more of a bar chart, a sector chart, and a rectangular chart. Different numerical bars in the bar chart indicate a frequency of use of different value ranges of the target protocol parameter. For example, the bar chart includes a plurality of numerical bars, and different numerical bars are used to indicate the frequency of use of different setting values or different value ranges of the target protocol parameter. Different regions of the sector chart indicate the frequency of use of different value ranges of the target protocol parameter. For example, the sector chart or the rectangular chart includes a plurality of regions, different regions from left to right correspond to the setting values of the target protocol parameter from small to great (or from great to small), and different regions are labeled with the frequency of use of the setting values corresponding to that region (which are labeled using colors, numbers, patterns, or other means). Forms of the parameter value distribution charts corresponding to different target protocol parameters may be the same or different.

In some embodiments, different types of the parameter value distribution charts are displayed on the graphical user interface of the terminal device, and the user performs a selection operation on a target type of the parameter value distribution chart on the graphical user interface. After receiving the selection operation of the user, the terminal device displays the parameter value distribution chart of the target type on the graphical user interface. Optionally, the parameter value distribution chart of a default type is displayed on the graphical user interface, and the user may change the type of the parameter value distribution chart. For example, the default type is the bar chart, and the user is able to change the default type to the sector chart.

The relative position refers to a position of the current setting value in the statistical distribution situation of the historical setting values. For example, as described above, the statistical distribution situation includes the reference value range, and the relative position reflects whether the current setting value is located within the reference value range. For another example, the statistical distribution situation includes the parameter value distribution chart. The processing device 120 may add an annotation to the parameter value distribution chart to indicate the relative position of the current setting value in the parameter value distribution chart. The annotation may include patterns, colors, arrows, lines, etc. The annotations corresponding to different types of parameter value distribution charts are in the same form or different forms.

Taking the bar chart as an example, the processing device 120 determines the numerical bar in which the current setting value is located, and annotates the numerical bar using the annotation. Optionally, the processing device 120 may annotate the numerical bar in which the current setting value is located using a different color than the other numerical bars. Alternatively, the processing device 120 may annotate the numerical bar in which the current setting value is located using an arrow or other patterns.

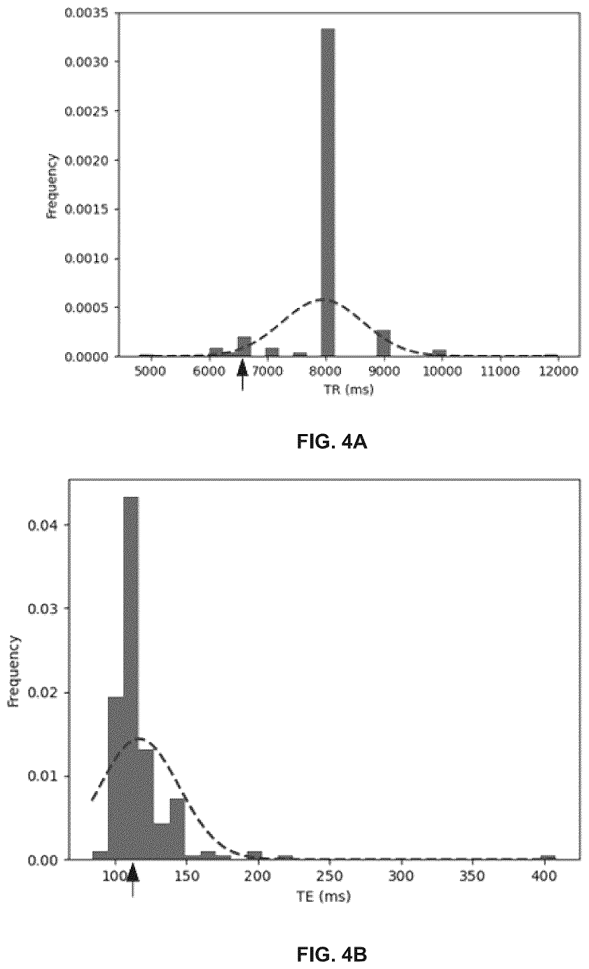

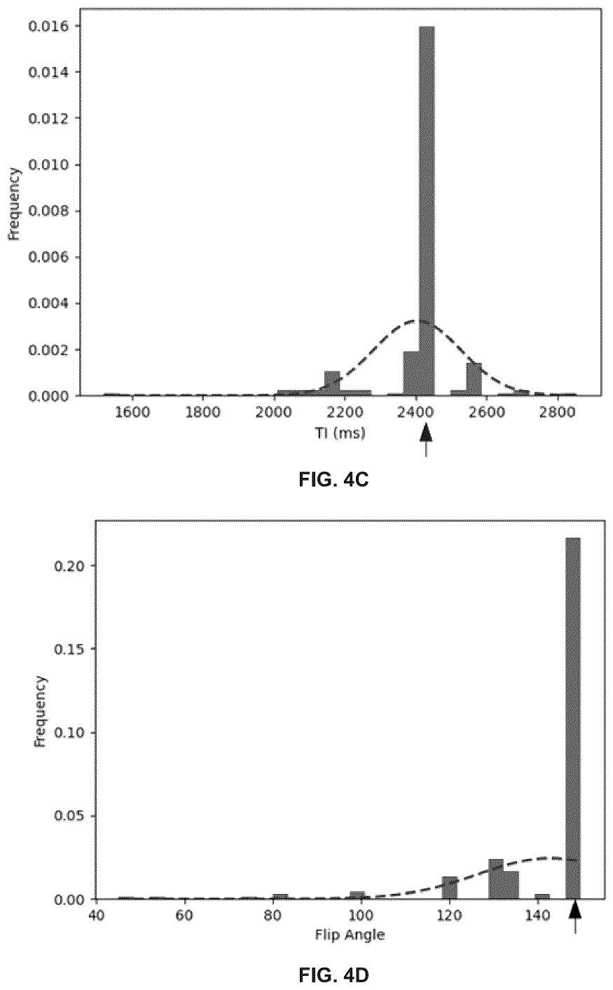

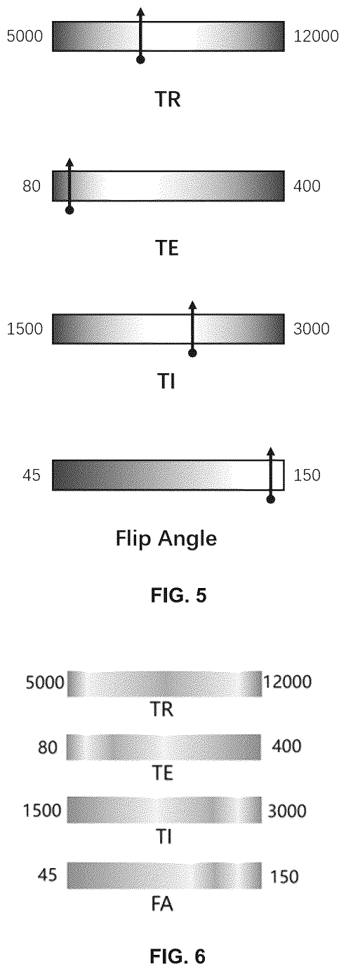

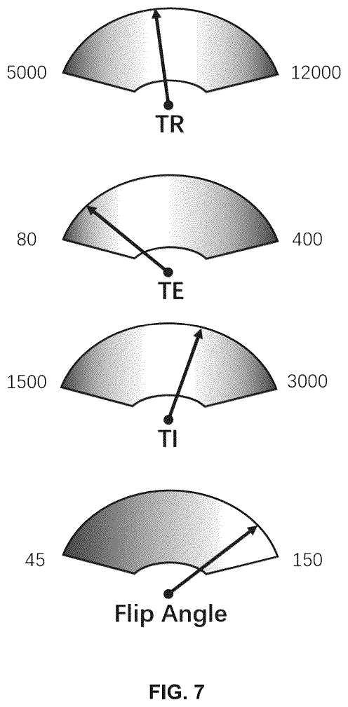

For exemplary purposes, FIGS. 4A-7 show schematic diagrams illustrating exemplary parameter value distribution charts of four key parameters: a TR, an echo time (TE), a time of inversion (TI), and a flip angle (FA) according to some embodiments of the present disclosure. The parameter value distribution charts shown in FIGS. 4A-4D are bar charts, the numerical bars pointed by arrows are the numerical bars where current setting values of the four key parameters are located. The parameter value distribution charts shown in FIG. 5 and FIG. 6 are rectangular charts. Positions of the arrows in FIG. 5 are positions of the current setting values of the four key parameters. FIGS. 5 and 6 use different colors to annotate how often different value ranges are used. A white color in FIG. 5 indicates a high frequency of use, and a black color indicates a low frequency of use. A white color in FIG. 6 indicates a low frequency of use, and a black color indicates a high frequency of use. The parameter value distribution charts shown in FIG. 7 are sector charts, the positions of the arrows are the positions of the current setting values of the four key parameters. FIG. 7 uses different colors to annotate the frequency of use of different value ranges, a white color indicates a high frequency of use, and a black color indicates a low frequency of use. The sector charts including pointers as the annotation may also be referred to as dashboard charts.

In some embodiments, the parameter value distribution charts corresponding to various types of reference scanning protocols are stored in the protocol database. Each protocol parameter of each type of reference scanning protocols has a corresponding parameter value distribution chart. The processing device 120 may search, in the protocol database, for the reference scan protocol corresponding to an identifier of the scanning protocol to be verified. The processing device 120 may further determine the parameter value distribution chart for the target protocol parameter based on the corresponding parameter value distribution chart of the searched reference scanning protocol.

The identifier of the scanning protocol is a unique identification of the scanning protocol. The identifier of the scanning protocol is set based on features such as a scanning protocol name, identification information (a gender, an age, etc.) of a scanning object, a scanning body part, a protocol label that identifies the scanning body part, and a sequence required for the scanning. For example, the identifier of the scanning protocol is the scanning protocol name. In some embodiments, the identifier of the scanning protocol is a unique number corresponding to the scanning protocol, e.g., each number corresponds to a single scanning protocol, and different scanning protocols have different numbers.

In some embodiments, the processing device 120 searches the reference scanning protocol in the protocol database corresponding to the identifier of the scanning protocol based on the identifier of the scanning protocol. The processing device 120 also determines the parameter value distribution chart corresponding to the searched reference scanning protocol as the parameter value distribution chart of the target protocol parameter and displays the parameter value distribution chart on the graphical user interface.

In this embodiment, after obtaining the scanning protocol determined by the user, the processing device 120 is able to determine the parameter value distribution chart by directly searching the protocol database, thereby improving an efficiency of obtaining the parameter value distribution chart. In addition, the parameter value distribution chart is obtained from the protocol database that stores a great number of historical scanning protocols, which is of higher utility and reliability, and is able to improve an accuracy of verifying the scanning protocol.

In 230, prompt information relating to parameter setting based on the relative position may be generated.

The prompt information refers to information used to indicate whether the current setting value is reasonable or not. The prompt information may further include other types of information provided for the user to refer to, such as the parameter value distribution chart added with the annotation, and a reference value range of the target protocol parameter.

In some embodiments, the prompt information is a graphic prompt, such as a box/a highlighting color, etc., on the graphical user interface to mark an unreasonable current setting value. For illustrative purposes only, a green color is used to label a reasonable current setting value, and a red color is used to label a unreasonable current setting value.

In some embodiments, the processing device 120 performs a reasonableness verification on the current setting value of the target protocol parameter based on the relative position to obtain a parameter verification result, the parameter verification result indicating whether the current setting value is reasonable; in response to determining that the parameter verification result indicates that the current setting value is unreasonable, the processing device 120 generates the prompt information, the prompt information including a reference parameter value of the target protocol parameter.

The reasonableness verification is used to verify whether the current setting value of the target protocol parameter is reasonable. Accordingly, the parameter verification result includes that the current setting value is reasonable, or the current setting value is unreasonable. The reasonableness verification for the current setting value of the parameter includes a parameter range verification and a parameter order verification. The parameter range verification refers to verifying whether the current setting value of the target protocol parameter is within the reference value range. The parameter order verification refers to verifying whether the order of parameter setting is reasonable. In some special situations, the protocol parameters in the scanning protocol need to be set in a specific order. When displaying the protocol parameters through a display device, the protocol parameters are displayed one by one according to the specific order.

In some embodiments, the processing device 120 performs the reasonableness verification on the current setting value according to a preset rule to obtain the parameter verification result of whether the current setting value is reasonable. For example, the processing device 120 determines whether the current setting value is null/0. If the current setting value of the target protocol parameter is null/0, the parameter verification result is that the current setting value is set unreasonably. If the current setting value of the target protocol parameter is not null/0, the parameter verification result is that the current setting value is set reasonably. In some embodiments, for the target protocol parameter whose current setting value is not null/0, the processing device 120 performs further reasonableness verifications (e.g., determining whether the current setting value is within the reference value range) using other verification manners to obtain the parameter verification result.

In some embodiments, the prompt information includes the parameter value distribution chart added with the annotation. For example, the prompt information includes the schematic diagrams as shown in FIG. 4A-FIG. 7.

In some embodiments, the relative position of the current setting value in the statistical distribution situation is determined based on the annotation in the parameter value distribution chart. For example, the relative position of the current setting value in the statistical distribution situation is the position indicated by the arrow annotation as shown in FIG. 4A-FIG. 7.

In some embodiments, the processing device 120 determines, based on the parameter value distribution chart with the annotation, whether the current setting value of the target protocol parameter is within the reference value range; in response to determining that the current setting value is in the reference value range, the processing device 120 determines that the parameter verification result is that the current setting value is reasonable; in response to determining that the current setting value is not within the reference value range, the processing device 120 determines that the parameter verification result is that the current setting value is unreasonable.

The reference value range refers to a range in which the setting values of the target protocol parameter are frequently used by the user. In some embodiments, the reference value range is determined based on the frequency of use of the historical setting values of the target protocol parameter. For example, the processing device 120 determines a range corresponding to the historical setting values that are used more frequently than a preset frequency threshold as the reference value range for the current setting value. The reference value ranges for the current setting value corresponding to different target protocol parameters may be the same or different. The processing device may compare the current setting value of each target setting parameter with the corresponding reference value range to determine whether the current setting value of the target protocol parameter is reasonable or not, and obtain the parameter verification result of the target protocol parameter.

In some embodiments of the present disclosure, by performing the reasonableness verification on the current setting value, and outputting the prompt information based on the verification result, a guide is provided for the user to adjust the target protocol parameter, so that the user is able to promptly clarify whether or not it is necessary to adjust the current setting value of the target protocol parameter before starting the scanning, thereby avoiding a waste of scanning time due to unreasonable setting of the target protocol parameter, and improving a scanning efficiency.

In some embodiments, when the parameter verification result is that the current setting value is unreasonable, the processing device outputs the current setting value of the target protocol parameter, the corresponding reference value range and/or the historical setting values of other users as the prompt information. For example, in response to the clicking or touching operation (e.g., a double-clicking operation) of the user, the processing device displays the unreasonably set current setting value of the target protocol parameter, and displays the reference value range determined based on the historical setting values and/or the historical setting values of other users, etc.

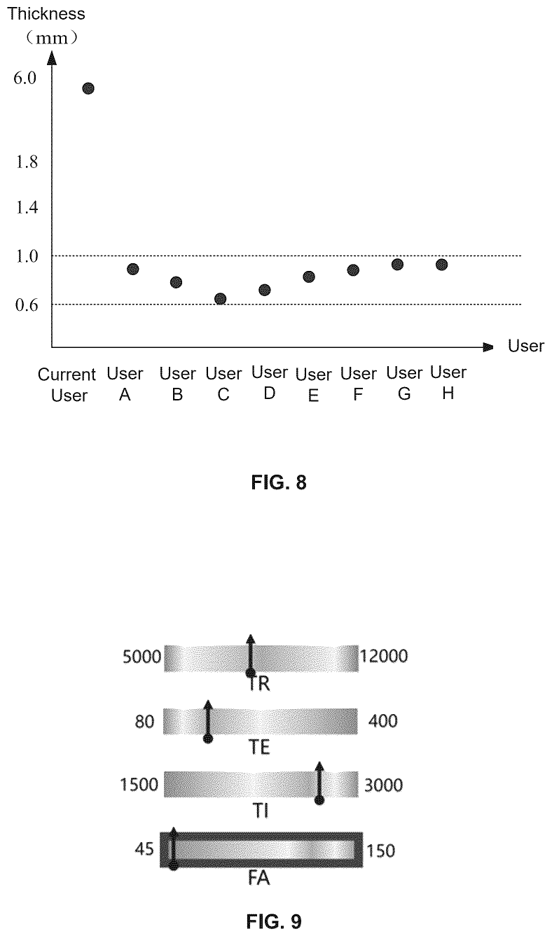

FIG. 8 is a schematic diagram illustrating a graphical user interface according to some embodiments of the present disclosure. Exemplarily, assuming that the target protocol parameter is the layer thickness in FIG. 3, in response to a double-click operation of a user on the layer thickness in the graphical user interface, the processing device 120 displays a reference value range (0.6-1.0) for the layer thickness, as well as other historical setting values used in historical scanning, in a form of a graph shown in FIG. 8. In some embodiments, the graphical user interface further displays a scanning performance indicator corresponding to each setting value (not shown). The scanning performance indicator may include at least one of an SNR, a contrast ratio, and a scanning time, etc. For a numerical setting value, a chart may be used to display the current setting value and the reference value range. For an optional setting value, text may be used to display the current setting value and an option content corresponding to the reference value range.

In some embodiments, in response to determining that the current setting value of a target protocol parameter is not within the reference value range, the processing device 120 determines a reference parameter value based on the reference value range. Prompt information further includes the reference parameter value. The reference parameter value refers to a recommended setting value of the target protocol parameter for reference to the user. In some embodiments, the prompt information also includes an advice for a parameter setting order under different parameter setting requirements. The different parameter setting requirements include the SNR, the contrast ratio, the scanning time, etc. The advice for the parameter setting order refers to an order of setting the SNR, the contrast ratio, or the scanning time, e.g., the SNR is set first, then the contrast ratio is set, and finally, the scanning time is set. In some embodiments, the reference parameter value is determined based on the reference value range. For example, the processing device 120 selects the setting value that is the most frequently used or an average setting value of the reference value range as the reference parameter value.

In some embodiments, the processing device 120 obtains profile information of a scanning object; and determines the reference parameter value of the target protocol parameter by inputting the profile information and the statistical distribution situation of the historical setting values of the target protocol parameter into a parameter recommendation model.

The profile information of the scanning object refers to data related to the scanning object itself or scanning requirements of the scanning object. For example, the profile information of the scanning object includes an age, a symptom, a scanning body part, a height, a weight, a medical history, etc. In some embodiments, the processing device 120 receives the profile information of the scanning object input by the user, or obtains the profile information of the scanning object from a profile database (e.g., based on a name, a number, etc. of the scanning object).

The parameter recommendation model refers to a machine learning model used to recommend the reference parameter value for the user. The parameter recommendation model may be a neural network model, a deep neural network model, etc. The input to the parameter recommendation model includes the profile information of the scanning object and the statistical distribution situation of the historical setting values of the target protocol parameter, and an output includes the reference parameter value of the target protocol parameter.

In some embodiments, the parameter recommendation model is obtained by training an initial model based on a second sample and a second label. The second sample includes sample profile information of a historical patient and a sample statistical distribution situation of the target protocol parameter, and the second label is a manually labeled label reference parameter value corresponding to the target protocol parameter of the historical patient.

In some embodiments of the present disclosure, the reference parameter value is given by a machine learning model based on a patient profile and the historical setting values, and the given reference parameter value is more in line with the current actual situation of the patient, making the parameter setting more reasonable and accurate, and improving a scanning efficiency and a scanning effect. When the current setting value of the target protocol parameter is unreasonable, the reference parameter value is provided to the user, thus improving the accuracy and efficiency of the protocol parameter setting, lowering requirements on the user, and reducing a workload of the user.

In some embodiments, after obtaining the current setting values of a plurality of protocol parameters of the scanning protocol, the processing device 120 inputs the current setting values of the plurality of protocol parameters of the scanning protocol into an image quality prediction model to obtain an image quality prediction result corresponding to the current setting values; determines whether the image quality prediction result satisfies a preset requirement; in response to determining that the image quality prediction result does not satisfy the preset requirement, the processing device 120 receives modified setting values of the plurality of protocol parameters from the user through a terminal device until the image quality prediction result corresponding to the modified setting values satisfies the preset requirement. In this way, the image quality of the scanning image may be predicted before scanning based on the current setting values of the plurality of protocol parameters, and if the image quality fails to satisfy the preset requirement, the current setting values of the protocol parameters may be quickly adjusted, which avoids a time cost for re-executing the scanning because the quality of the scanning image does not satisfy the requirement. The preset requirement may be the same requirement as described in operation 220.

In some embodiments, when the image quality prediction result does not satisfy the preset requirement, it indicates that the protocol parameters need to be adjusted. The processing device 120 may display the prompt information to the user through the terminal device to remind the user to adjust the scanning parameters. In some embodiments, the prompt information also includes reference parameter values of the protocol parameters. The user may modify the setting values of the protocol parameters based on the reference parameter values, and the processing device 120 may receive the modified setting values input by the user through the terminal device and input the modified setting values into the image quality prediction model to obtain the image quality prediction result corresponding to the modified setting values. The processing device 120 may further determine whether the image quality obtained by scanning satisfies the preset requirement based on the image quality prediction result. By repeating the foregoing process until the image quality prediction result corresponding to the modified setting values satisfies the preset requirement, the accuracy of the scanning parameter setting of the user may be ensured.

The prompt information may be displayed anywhere in the graphical user interface. Exemplarily, the processing device 120 displays the prompt information at a position in the graphical user interface where the current setting value is located. In some embodiments, when the current setting value is abnormal, a parameter value distribution chart corresponding to the current setting value may be marked and displayed in the graphical user interface of the terminal device. For example, the parameter value distribution chart corresponding to the current setting value is highlighted. As shown in FIG. 9, the current setting value of the target protocol parameter FA is abnormal, and the rectangular box of the rectangular chart corresponding to the target protocol parameter FA is bolded.

In some embodiments, the scanning protocol includes a plurality of protocol parameters, and the parameter value distribution charts of the plurality of protocol parameters are displayed in a preset sequence, the preset sequence being determined based on an importance of the plurality of protocol parameters to the imaging quality. For example, the protocol parameters are sorted in a descending sequence according to their importance to the imaging quality, i.e., the parameter value distribution chart corresponding to the protocol parameter with the highest importance is displayed first, and the parameter value distribution chart corresponding to the protocol parameter with the lowest importance is displayed last.

The importance of a protocol parameter to the imaging quality refers to a magnitude of an effect of the protocol parameter on the quality of the scanning image. The greater the importance of the protocol parameter to the imaging quality, the greater the impact on the quality of the scanning image. To put it another way, the importance of the protocol parameter to the imaging quality is reflected in a magnitude of the change in the quality of the scanning image when the content of the protocol parameter changes. For example, if a protocol parameter is of a high importance to the imaging quality, a small change in the protocol parameter (e.g., a value changes by 0.5, 1, 3, etc.) results in a great change in the quality of the scanning image (e.g., a reduction in an overall quality score of the scanning image from a high quality to a medium-high quality).

In some embodiments, the importance of a protocol parameter to the imaging quality is expressed in terms of an importance coefficient. The higher the importance coefficient of a protocol parameter, the higher the importance of the protocol parameter to the imaging quality, and the higher the sequence of the parameter value distribution chart for the protocol parameter is displayed.

In some embodiments, the processing device 120 determines a scanning body part of a scanning object; obtains historical scanning images corresponding to the scanning body part and historical values of the protocol parameters corresponding to each historical scanning image; determines the importance coefficient for each of the protocol parameters based on the historical scanning images and the historical values of the protocol parameters; and determines the preset sequence based on the importance coefficient of each of the protocol parameters. The processing device 120 determines the scanning body part of the scanning object based on medical data of the scanning object and/or a scannable site corresponding to the scanning protocol.

In some embodiments, the processing device 120 may determine an image quality assessment result of each historical scanning image, and determine a correlation between each protocol parameter and the imaging quality based on the image quality assessment result of each historical scanning image. The higher the correlation, the higher the importance coefficient. For example, the processing device 120 determines the correlation between the protocol parameter and the imaging quality by a data analysis method (e.g., a linear regression analysis, a multiple regression analysis method, etc.) and further determines the importance coefficient for each protocol parameter.

In some embodiments, for each historical scanning image corresponding to the scanning body part, the processing device 120 randomly adjusts the historical values of the protocol parameters corresponding to the historical scanning image to obtain adjusted values of the protocol parameters; inputs the adjusted values into an image quality prediction model to obtain an image quality prediction result; determines an image quality difference between an image quality assessment result of the historical scanning image and the image quality prediction result; and determines the importance coefficient for each protocol parameter based on the image quality difference corresponding to each historical scanning image.

The random adjustment refers to randomly adjusting numerical values of the historical setting values of the protocol parameters. For example, the setting values are randomly increased or decreased. In some embodiments, the random adjustment is an adjustment on the historical setting values of a portion of the plurality of protocol parameters, e.g., there are a total of 5 protocol parameters, and the random adjustment is the adjustment of one, or a few (e.g., 2, 3) of the 5 protocol parameters. In some embodiments, the random adjustment is the adjustment for all of the plurality of protocol parameters, e.g., the random adjustment is the adjustment on all 5 protocol parameters.

In some embodiments, the historical setting values of the plurality of protocol parameters are adjusted according to a control variable manner to better determine the effect of the protocol parameters on the imaging quality. For example, by adjusting only one protocol parameter for each random adjustment and controlling the historical setting values of the other protocol parameters to remain unchanged, a magnitude of impact of the adjusted protocol parameter on the imaging quality is determined, and then the importance coefficient corresponding to that protocol parameter is determined.

In some embodiments, the processing device predicts the quality of the scanning image based on the adjusted setting values of the plurality of protocol parameters using the image quality prediction model, and in this way, the process of determining the importance coefficient is simplified. The image quality prediction model is a machine learning model for predicting, based on the setting values of the protocol parameters, the quality of the scanning image obtained by scanning using the setting values of the protocol parameters. In some embodiments, the image quality prediction model is obtained by training an initial model based on a third sample and a third label. The third sample includes sample setting values of the protocol parameters, and the third label is a manually labeled image quality assessment result of a scanning image corresponding to the sample setting values. For example, the third sample may be setting values of the protocol parameters corresponding to a historical scanning image, and the third label may be the image quality assessment result of the historical scanning image labeled by an expert. The image quality prediction model may be trained by common model training methods (e.g., the gradient descent method, etc.). In some embodiments,

In some embodiments, if a certain protocol parameter is adjusted, the greater the image quality difference between the image quality assessment result of a historical scanning image and the image quality prediction result, the greater the impact of the protocol parameter on the imaging quality, and correspondingly, the greater the importance coefficient of the protocol parameter. A correspondence between the image quality difference and the importance coefficient of the protocol parameter may be preset. For example, a mapping relationship between the image quality difference and the importance coefficient of a protocol parameter is established. Merely by way of example, the image quality difference is expressed as a score difference between an image quality assessment score and an image quality prediction score, the importance coefficient of the protocol parameter is expressed by a numerical value, and the mapping relationship is a corresponding relationship between the score difference and the numerical value of the importance coefficient.

Exemplarily, it is assumed that a historical scanning image corresponds to 4 protocol parameters. For each protocol parameter, the processing device 120 randomly adjusts the historical setting value 4 times (the remaining 3 protocol parameters are unchanged) to obtain 4 sets of adjusted values of the protocol parameters, inputs the adjusted values of the 4 sets of protocol parameters into the image quality prediction model respectively, and obtains 4 image quality prediction results. The processing device 120 further determines the image quality difference between the image quality assessment result of the historical scanning image and each of the 4 image quality prediction results to obtain 4 image quality differences. The final image quality difference may be the sum or the average of the 4 image quality differences. According to the mapping relationship between the image quality difference and the importance coefficient, the importance coefficient of the corresponding protocol parameter is determined.

In some embodiments of the present disclosure, by determining the sequence of the parameter value distribution chart based on the importance of the protocol parameters to the imaging quality, the user is able to quickly focus on important protocol parameters, and the experience of the parameter setting of the user is improved. In addition, determining the importance of each protocol parameter with an aid of the image quality prediction model reduces an influence of manual subjective factors and improves the accuracy of the importance assessment.

In some embodiments, the processing device 120 determines an associated protocol parameter that has an association with the target protocol parameter; determines a parameter value of an association relationship parameter based on the current setting value of the target protocol parameter and a second current setting value of the associated protocol parameter; and performs a reasonableness verification on the current setting value of the target protocol parameter and the second current setting value of the associated protocol parameter based on the parameter value of the association relationship parameter and a second statistical distribution situation corresponding to the association relationship parameter.

The associated protocol parameter refers to a protocol parameter that has a parameter association with the target protocol parameter. The parameter association refers to an existence of a certain connection between protocol parameters. For example, by combining the protocol parameters that have a certain connection, another protocol parameter that is not directly included in the scanning protocol is obtained. For example, in the scanning protocol, a layer number, a layer thickness, and a layer spacing are three parameters that together affect a scanning field of view size, and if the target protocol parameter is the layer number, the layer thickness and the layer spacing are the associated protocol parameters of the target protocol parameter. Combining the layer number, the layer thickness, and the layer spacing, the FOV, is obtained.

The association relationship parameter may be a combination parameter obtained by combining the target protocol parameter and the associated protocol parameter, or a protocol parameter reflecting the association between the target protocol parameter and the associated protocol parameter.

The second current setting value refers to a current setting value of the associated protocol parameter. In some embodiments, the processing device 120 obtains the second current setting value similarly to obtaining the current setting value.

The second statistical distribution situation refers to a distribution situation of historical values of the association relationship parameter. In some embodiments, the processing device 120 determines the second statistical distribution situation similarly to determining the statistical distribution situation corresponding to the target protocol parameter. It should be understood that as the association relationship parameter is not directly embodied in a historical scanning protocol, the historical value of the association relationship parameter needs to be calculated based on historical setting values of the target protocol parameter and the associated protocol parameter in the historical scanning protocol.

In some embodiments, the processing device 120 performs a reasonableness verification on the association relationship parameter based on the second statistical distribution situation to further determine, whether the current setting value of the target protocol parameter and/or the second current setting value of the associated protocol parameter is reasonable. The manner of performing the reasonableness verification on the association relationship parameter based on the second statistical distribution situation is similar to the manner of performing the reasonableness verification on the target protocol parameter in operation 230.

In some embodiments, if a result of the reasonableness verification on the association relationship parameter is an unreasonable result, it is possible that the current setting value of the target protocol parameter is unreasonable and/or the second current setting value of the associated protocol parameter is unreasonable. The processing device 120 may control the terminal device to issue prompt information indicating that the current setting value of the target protocol parameter is unreasonable and/or the second current setting value of the associated protocol parameter is unreasonable. The user may adjust the current setting value of the target protocol parameter and/or the second current setting value of the associated protocol parameter according to the prompt information, and perform the reasonableness verification on the adjusted setting value of the target protocol parameter and/or the adjusted setting value of the associated protocol parameter. Further, the reasonableness verification may be performed on the association relationship parameter corresponding to the adjusted setting value of the target protocol parameter and/or the adjusted setting value of the associated protocol parameter. The user may repeat the above process until the result of the reasonableness verification on the association relationship parameter is reasonable.

In some embodiments of the present disclosure, by performing the reasonableness verification on the association relationship parameter corresponding to the target protocol parameter and the associated protocol parameter, the reasonableness verification can be performed on an implicit associated relationship between the plurality of protocol parameters of the scanning protocol. This further reduces the reliance on operator experience and improves the efficiency and accuracy of the protocol parameter setting.

In some embodiments, the processing device 120 generates a target scanning protocol based on the scanning protocol; uploads the target scanning protocol and the identifier of the target scanning protocol to an ecological database for protocol verification; and in response to determining that the target scanning protocol passes the protocol verification, saves the target scanning protocol and the identifier of the target scanning protocol into the protocol database.

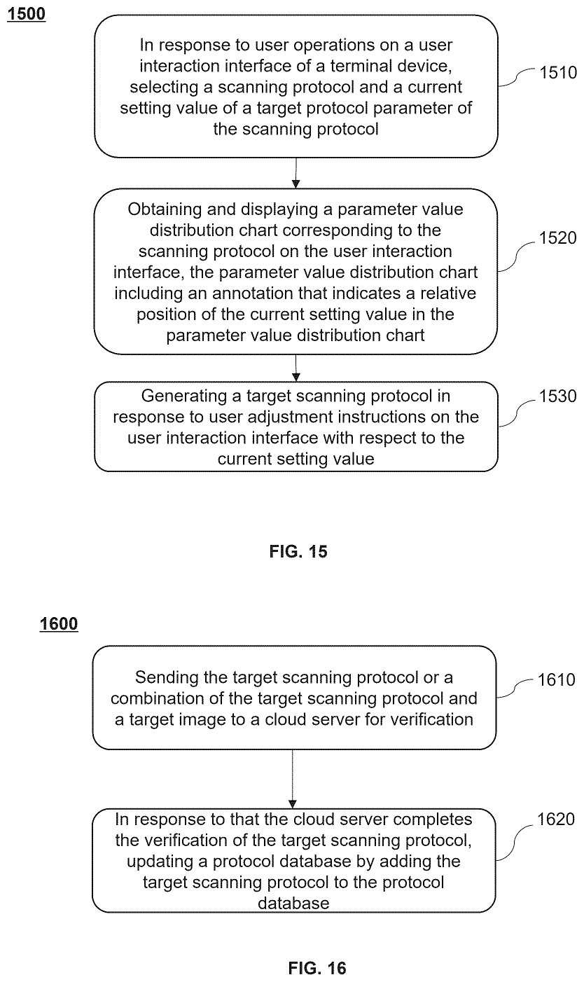

The target scanning protocol refers to a scanning protocol whose all target protocol parameters pass the reasonableness verification. The target scanning protocol may be executed on the scanning object in the scan of the scanning object. For example, if all the target protocol parameters in the scanning protocol pass the reasonableness verification, the scanning protocol is directly designated as the target scanning protocol. More descriptions about generating the target scanning protocol may be found in FIG. 15 and the related descriptions.

The ecological database may be a shared database which is accessible by different users (e.g., scanning technologists in different hospitals). The ecological database may be used to store the scanning protocols to be verified by managers (e.g., the target scanning protocols uploaded to the ecological database). In some embodiments, the ecological database and the protocol database are the same database or different databases. In some embodiments, the ecological database may be stored in a cloud server as described in connection with FIG. 16.

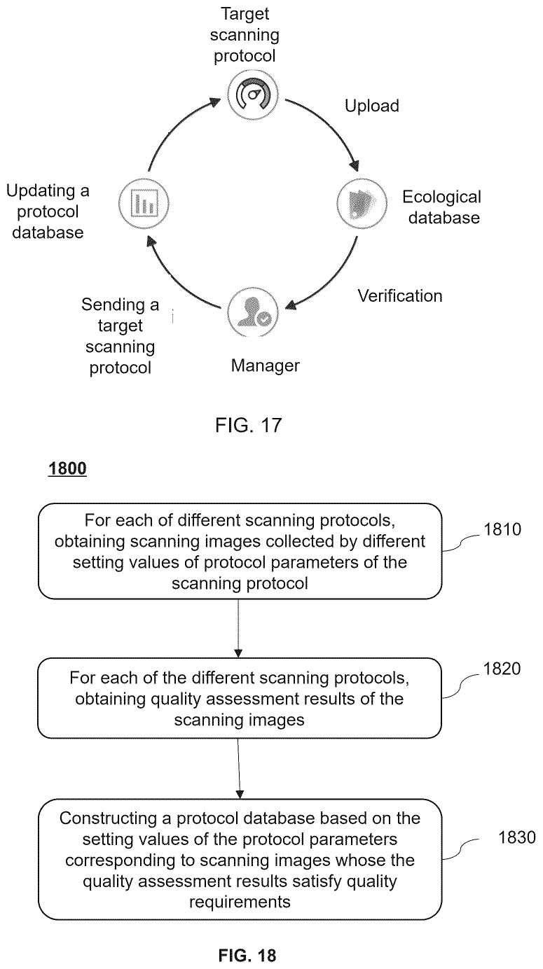

In some embodiments, after the target scanning protocol and the identifier thereof are uploaded to the ecological database, the manager verifies the target scanning protocol to determine whether the target scanning protocol is appropriate. For example, the manager verifies whether a scanning image obtained by scanning with the target scanning protocol satisfies requirements (the scanning image corresponding to the target scanning protocol is uploaded together to the ecological database for verification by the manager). If the target scanning protocol passes the verification, the target scanning protocol and the identifier thereof are uploaded into the protocol database. If there is already a record of the target protocol parameter stored in the protocol database, the target scanning protocol and the corresponding parameter value distribution charts in the protocol database may be updated. More descriptions about updating the protocol database may be found in FIG. 16, FIG. 17 and their related descriptions.

In some embodiments of the present disclosure, the protocol database stores reference contents of each protocol parameter corresponding to different scanning protocols, which provides a verification standard for the reasonableness verification, and improves the comprehensiveness and reliability of the reasonableness verification. Additionally, by providing the prompt information and the reference parameter value when the user sets the protocol parameters, a professional requirement on the user in the protocol parameter setting process is reduced, which improves the efficiency and the accuracy of protocol parameter setting.

FIG. 10 is a flowchart illustrating an exemplary process for determining a scanning protocol to be verified according to some embodiments of the present disclosure. In some embodiments, a process 1000 is performed by a processing device (e.g., the processing device 120, etc.). In some embodiments, the process 1000 is performed prior to operation 210 of the process 200. As shown in FIG. 10, the process 1000 includes the following operations.

In 1010, a selected scanning protocol group for a current scanning scene may be obtained.

The current scanning scene refers to a scanning scene of a target scanning to be performed. The current scanning scene may indicate a scanning requirement.

There are various manners to determine the current scanning scene. In some embodiments, the current scanning scene is determined by: obtaining profile information of the scanning object, and determining the current scanning scene based on the profile information. Alternatively, the current scanning scene is determined by: obtaining at least one protocol label based on the at least one selected scanning protocol included in the selected scanning protocol group, and determining the current scanning scene based on the at least one protocol label, the at least one protocol label being used to identify a scanning body part.

In some embodiments, the processing device 120 reads the protocol label of the scanning protocol(s) included in the selected scanning protocol group and determines a scanning body part corresponding to the scanning protocol(s) in the selected scanning protocol group according to a correspondence between the protocol label and the scanning body part, thereby determining the scanning scene corresponding to the scanning body part as the current scanning scene. Each scanning protocol may be preset with a corresponding protocol label. For example, a scanning protocol “T2_fse_tra” is preset with a corresponding protocol label “head-hippocampus,” then based on the protocol label “head-hippocampus,” the processing device 120 determines that the current scanning scene is a scene in which the hippocampus of the head is scanned. In this way, the current scanning scene may be determined even when the profile information of the scanning object is not obtained.

The selected scanning protocol group refers to a collection of scanning protocols that are selected for the target scanning. In some embodiments, the selected scanning protocol group includes at least one selected scanning protocol that is selected by the user.

In some embodiments, the processing device 120 obtains the scanning protocol(s) selected by the user at a graphical user interface as the selected scanning protocol group of the current scanning scene. Exemplarily, the user selects a scanning protocol 1, a scanning protocol 2, and a scanning protocol 3 in a protocol selection region of the graphical user interface, and the processing device 120 reads the scanning protocols 1-3 as the selected scanning protocol group of the current scanning scene. The processing device 120 obtains the scanning protocols selected by the user based on a touchscreen operation, a keyboard and mouse operation, a remote control operation, etc. of the user to form the selected scanning protocol group.

For example, the user inputs keywords such as a number, a name, a function, etc., of the scanning protocol, and the processing device 120 filters the scanning protocols that match the keywords from the protocol database and generates a protocol list for display. The processing device 120 obtains the scanning protocols selected by the user based on the selection operation of the user in the protocol list. The selected scanning protocol group may be empty if the current user does not select any scanning protocol.

It is understood that the scanning protocols in the selected scanning protocol group may be scanning protocols that are used on the scanning objects when performing the target scanning.

In 1020, a recommended scanning protocol group corresponding to the current scanning scene may be determined. In some embodiments, the recommended scanning protocol group includes at least one recommended scanning protocol.

The recommended scanning protocol group is a scanning protocol group that satisfies the scanning requirement of the current scanning scene. For example, the processing device 120 determines, through an industry database, the recommended scanning protocol group corresponding to the current scanning scene. Specifically, the industry database may store scanning protocol groups used by different scanning scenes, and the scanning protocol group corresponding to the current scanning scene in the industry database may be used as the recommended scanning protocol group. The industry database and the protocol database may be the same database or different databases.

For another example, the processing device 120 determines a scanning protocol group that matches the current scanning scene as the recommended scanning protocol group based on a matching relationship between scanning scenes and scanning protocol groups. Exemplarily, a current scanning scene A corresponds to a recommended scanning protocol group (including the scanning protocol 2, the scanning protocol 3, a scanning protocol 4).

In some embodiments, the recommended scanning protocol group is determined based on a usage rate of scanning protocols in the current scanning scene. For example, the processing device 120 obtains sample scanning protocol groups used by a plurality of reference users under the current scanning scene, and determines the recommended scanning protocols whose user usage rate is higher than a usage rate threshold, and these recommended scanning protocols form the recommended scanning protocol group. The sample scanning protocol group used by each reference user includes at least one scanning protocol. Optionally, the plurality of reference users described above may be users whose ranks are higher than a preset rank, and/or users whose hospitals belong to hospitals whose ranks are higher than a preset rank. Exemplarily, the scanning protocol group used by a physician at a director level and above of a tertiary hospital is selected as a sample scanning protocol group.

Alternatively, for a specific scanning protocol, a number of the sample scanning protocol groups containing the scanning protocol (i.e., a number of users using the scanning protocol) is determined, and a ratio of the number to a total number of the sample scanning protocol groups (i.e., a total number of users) is the user usage rate of the scanning protocol.

The usage rate threshold may be used to evaluate whether a scanning protocol is a common protocol for the current scanning scene. If the user usage rate of the scanning protocol is higher than the usage rate threshold, it indicates that the scanning protocol is a commonly used scanning protocol for the current scanning scene, and at this time, the scanning protocol may be used as a recommended scanning protocol. On the other hand, if the user usage rate of the scanning protocol is lower than the usage rate threshold, it indicates that the scanning protocol is not commonly used in the current scanning scene, and the scanning protocol may not be used as the recommended scanning protocol.

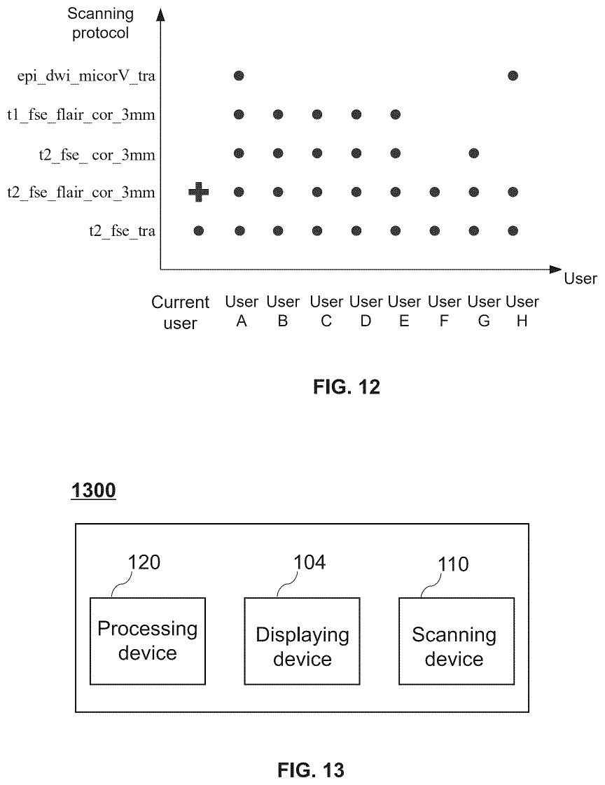

For example, refer to FIG. 12, which is a schematic diagram illustrating a graphical user interface according to some embodiments of the present disclosure. For a same scanning scene “scanning a hippocampus,” users A-H use a scanning protocol “t2_fse_flair_cor_3 mm,” a user usage rate of this scanning protocol is 100%, which is greater than a usage rate threshold of 95%, and the scanning protocol “12_fse_flair_cor_3 mm” is used as a recommended scanning protocol. Users A-E and a user G all use a scanning protocol “t2_fse_cor_3 mm,” and the user usage rate of this scanning protocol is 75%, which is less than the usage rate threshold of 95% so the scanning protocol “t2_fse_cor_3 mm” is not used as the recommended scanning protocol. By traversing each scanning protocol, the recommended scanning protocols included in the recommended scanning protocol group may be determined as: “t2_fse_flair_cor_3 mm” and “t2_fse_tra.” t2 indicates that a fluid has a high signal, fse denotes a fast spin echo (FSE), flair denotes a fluid attenuated (Inhibited) inversion recovery sequence (FLAIR), cor denotes a sagittal position, tra denotes a horizontal axis position, and 3 mm indicates a thickness.

In some embodiments, after determining the recommended scanning protocol group, the recommended scanning protocol group is displayed to prompt the current user. The recommended scanning protocol group may be displayed in a form of text in a configuration interface of the scanning protocol, or it may be displayed in other interfaces, in other styles, and the embodiments of the present disclosure do not limit that.

It may be understood that the value of the above usage rate threshold can be adjusted according to the actual situation. In some embodiments of the present disclosure, the usage rate threshold is related to a scanning frequency of the current scanning scene. Exemplarily, if the scanning frequency of the current scanning scene is higher, it indicates that the current user is more familiar with the current scanning scene, and accordingly, the usage rate threshold is set higher for displaying apparently missing scanning protocols. If the scanning frequency of the current scanning scene is lower, it indicates that the current user is less familiar with the current scanning scene, and accordingly, the usage rate threshold is set lower for displaying all possible scanning protocols of the current scanning scene, so as to help the current user to understand the relevant information.

Additionally, considering that the determined recommended scanning protocols may not be able to satisfy a scanning requirement, the processing device 120 may filter the recommended scanning protocols based on the needs of the scanning requirement. For example, after determining the recommended scanning protocols whose user usage rates are higher than the usage rate threshold, the processing device 120 obtains limitation information for the target scanning and filters out recommended scanning protocols that do not satisfy the limitation information. The limitation information includes a maximum scanning duration of the target scanning, a maximum resolution of a scanning image obtained by the target scanning, etc. Taking the maximum resolution of the scanning image as an example, if the resolution corresponding to a recommended scanning protocol is greater than the maximum resolution, the recommended scanning protocol is screened out. Taking a minimum resolution of the scanning image as an example, if the recommended scanning protocol is smaller than the minimum resolution, then the recommended scanning protocol is screened out. Taking the maximum scanning duration of the target scanning as an example, if a scanning duration required to complete all the scanning protocols after combining the recommended scanning protocol and the scanning protocols in the selected scanning protocol group is greater than the maximum scanning time, the recommended scanning protocol is screened out.

Further example, after determining the recommended scanning protocols whose user usage rates are higher than the usage rate threshold, the processing device 120 obtains historical scanning protocols used by the scanning object and screens out recommended scanning protocols that are the same as the historical scanning protocols. The historical scanning protocols refer to scanning protocols used by the scanning object during past scanning, which are, for example, the scanning protocols used in a last visit of the scanning object. The historical scanning protocols are obtained based on analysis of the past scanning images, or are obtained based on the profile information of the scanning object. Since the historical scanning protocols are scanning protocols that are already used to scan the scanning object, recommended scanning protocols that are the same as the historical scanning protocols are screened out, thereby reducing the scanning duration of the scanning object to improve a visit experience.

In 1030, the scanning protocol may be selected from the selected scanning protocol group and the recommended scanning protocol group and the scanning protocol may be displayed. The selected scanning protocol refers to the scanning protocol to be verified.

In some embodiments, after obtaining the recommended scanning protocol group corresponding to the current scanning scene, the processing device 120 compares the scanning protocols in the selected scanning protocol group and the recommended scanning protocol group, determines whether the selected scanning protocol group matches the current scanning scene. If the selected scanning protocol group does not match the current scanning scene, the processing device 120 obtains the unmatched scanning protocol in the selected scanning protocol group and the recommended scanning protocol group as the scanning protocol to be verified and displays the unmatched scanning protocols through the terminal device. The unmatched scanning protocol in the selected scanning protocol group and the recommended scanning protocol group includes a scanning protocol that is included in the selected scanning protocol group but not included in the recommended scanning protocol group, a scanning protocol that is included in the recommended scanning protocol group but not included in the selected scanning protocol group.

Exemplarily, if the scanning protocols in the selected scanning protocol group and the recommended scanning protocol group are the same, the processing device 120 determines that the selected scanning protocol group matches the current scanning scene. If the scanning protocols in the selected scanning protocol group and the recommended scanning protocol group are not the same, the processing device 120 determines that the selected scanning protocol group does not match the current scanning scene. The processing device 120 may also determine that the selected scanning protocol group matches the current scanning scene if the selected scanning protocol group includes all of the scanning protocols in the recommended scanning protocol group.

In some embodiments of the present disclosure, by comparing the user-selected scanning protocols and the recommended scanning protocol group, the current user is reminded of which mainstream scanning protocols are used in the industry for the current scanning scene, which helps the user in performing a scanning protocol selection decision. In order to enable the user to see the mainstream scanning protocols in a timely and intuitive manner, the unmatched scanning protocol may be displayed to the user to help the user add missing scanning protocols to the selected scanning protocol group or remove inappropriate scanning protocols from the selected scanning protocol group, thereby enhancing the rationality of scanning protocol selection and improving scanning reliability.

In some embodiments, the processing device 120 displays the scanning protocol to be verified using a different display manner than the other scanning protocols within the selected scanning protocol group. Exemplarily, referring to FIG. 3, the scanning protocol to be verified is displayed at the bottom of a protocol selection region in the graphical user interface, or the scanning protocol to be verified is displayed in a form of a pop-up as shown in FIG. 11.

In some embodiment of the present disclosure, the scanning protocol to be verified is determined automatically by comparing the selected scanning protocol group and the recommended scanning protocol group corresponding to the current scanning scene. The scanning protocol to be verified includes unmatched scanning protocol(s) in the selected scanning protocol group and the recommended scanning protocol group. In this way, before verifying the protocol parameters, the selected scanning protocol group is also verified, which realizes a double verification on the scanning protocols, assists the user in the scanning protocol selection and helps to improve the efficiency and convenience of the scanning protocol selection, and enhances the user experience.

FIG. 13 is a schematic diagram illustrating a system for setting protocol parameters according to some embodiments of the present disclosure. As shown in FIG. 13, the system 1300 for setting protocol parameters includes the processing device 120, a display device 104, and the scanning device 110. In some embodiments, the processing device 120 and the display device 104 are integrated into the scanning device 110 as a processing unit and a display unit, respectively.

The display device 104 may be configured to display a current setting value of the target protocol parameter in a graphical user interface.

The processing device 120 may be configured to obtain the current setting value of the target protocol parameter, perform a reasonableness verification on the current setting value to obtain a parameter verification result, and determine prompt information based on the parameter verification result.

In some embodiments, the display device 104 is also configured to display the prompt information in the graphical user interface to remind a user to adjust the target protocol parameter.

In some embodiments, the display device 104 is further configured to display a selected scanning protocol group in the graphical user interface before displaying the target protocol parameter in the graphical user interface. The selected scanning protocol group includes one or more selected scanning protocols selected by the user.

In some embodiments, the processing device 120 is also configured to determine whether the selected scanning protocol group matches a current scanning scene, and if the selected scanning protocol group does not match the current scanning scene, the processing device 120 is configured to generate navigation information, and generate a target protocol group that matches the current scanning scene in response to an operation of the user to adjust the selected scanning protocol group in the graphical user interface according to the navigation information. The navigation information is used to indicate an adjustment recommendation for the selected scanning protocol group.

In some embodiments, the navigational information includes at least one of a scanning protocol to be added to the selected scanning protocol group, a scanning protocol to be changed in the selected scanning protocol group and a corresponding changed scanning protocol, and a scanning protocol to be deleted from the selected scanning protocol group.

In some embodiments, the scanning device 110 is configured to execute the target protocol group on the scanning object under a control of the processing device 120.

In some embodiments, the above algorithm for determining whether the selected scanning protocol group matches the current scanning scene is a classification-based manner, such as a decision tree classification. The algorithm divides data of a database into a number of categories, with each category representing a type of similar data, and then a decision tree is used to categorize the individual data, so as to derive the category to which each data belongs, and thus calculate differences between the individual data and group data.

In some embodiments, the above algorithm for determining whether the selected scanning protocol group matches the current scanning scene is a manner based on a feature selection, which selects the most representative features from the group data, and then compares the individual data with these features, so as to derive the difference between the individual data and the group data.

In some embodiments, the determining whether the selected scanning protocol group matches the current scanning scene includes: obtaining the protocol database; the protocol database including a plurality of units, each unit including a historical scanning scene and corresponding plurality of historical scanning protocol groups; matching the protocol database according to the current scanning scene to determine the unit that matches the current scanning scene; separately calculating a matching degree between the selected scanning protocol group and each of a plurality of historical scanning protocol groups in the unit that matches the current scanning scene; according to the matching degree of the selected scanning protocol group and the plurality of historical scanning protocol groups, determining a reasonableness degree of the selected scanning protocol group under the current scanning scene; and, in response to that the reasonableness degree is within a set range, determining that the selected scanning protocol group matches the current scanning scene; or, in response to that the reasonableness degree exceeds the set range, determining that the selected scanning protocol group does not match the current scanning scene.

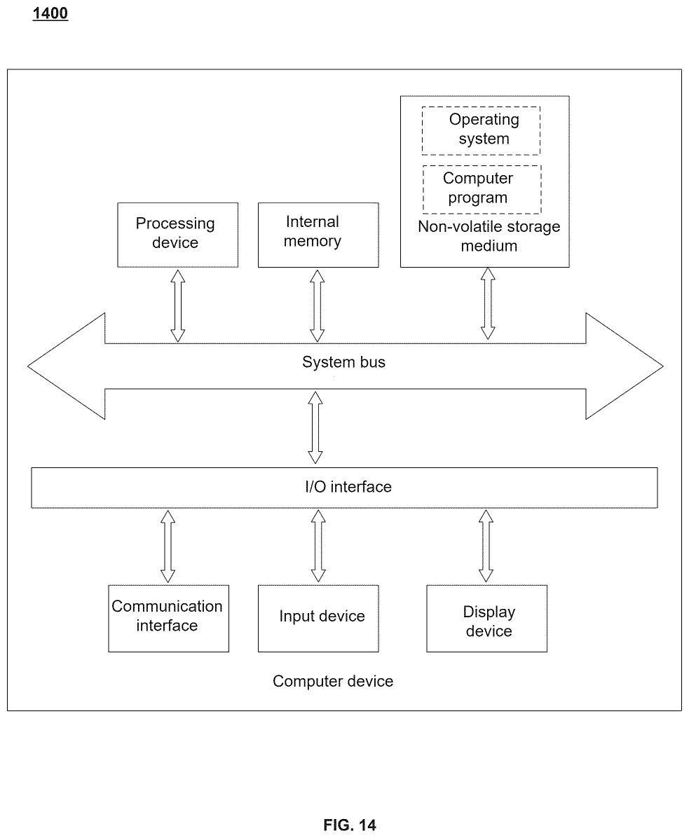

FIG. 14 is a schematic diagram illustrating a computer device according to some embodiments of the present disclosure. In some embodiments, a computer device 1400 is used to implement the processing device 120.

As shown in FIG. 14, the computer device 1400 includes a processing device (e.g., the processing device 120), a memory, a communication interface, a display device, and an input device connected through a system bus. The processing device of the computer device is used to provide computing and control capabilities. The memory of the computer device includes a non-volatile storage medium and an internal memory. The non-volatile storage medium stores an operating system and a computer program. The internal memory provides an environment for operation of the operating system and the computer program in the non-volatile storage medium. The communication interface of the computing device is used to communicate with external terminal devices in a wired or wireless mode, which is realized through WIFI, mobile cellular networks, near field communication (NFC), or other technologies. The computer program is executed by a processing device to implement a scanning protocol determination method. The display device of the computer device may be a liquid crystal display (LCD) or an e-ink display, and the input device of the computer device may be a touch layer overlaying the display device, or a computer device housing provided with keys, a trackball or a touchpad, and may be an external keyboard, a touchpad or a mouse, etc.

Those skilled in the art may understand that the structure illustrated in FIG. 14 is only a block diagram of a part of the structure related to the technical solution disclosed herein, which does not constitute a limitation on the computer device to which the solutions of the present disclosure are applied, and specific computer device may include more or fewer components than shown in the drawings, or combine certain components, or have a different arrangement of components.

FIG. 15 is a flowchart illustrating an exemplary process for generating a target scanning protocol according to some embodiments of the present disclosure. In some embodiments, a process 1500 is performed by the processing device 120. In some embodiments, the processing device 120 is a part of a terminal device.

In 1510, in response to user operations on a graphical user interface of the terminal device, a scanning protocol and a current setting value of a target protocol parameter of the scanning protocol are selected.

More descriptions about the scanning protocol and the current setting value may be found in the descriptions of operation 210 in FIG. 2.

In 1520, a parameter value distribution chart corresponding to the scanning protocol is obtained and displayed in the graphical user interface, the parameter value distribution chart having an annotation that indicates a relative position of the current setting value in the parameter value distribution chart. For example, the parameter value distribution chart is a dashboard chart, which is used to characterize a health (or fitness) of the current setting value.

After obtaining the parameter value distribution chart corresponding to the scanning protocol, the processing device displays the parameter value distribution chart in the graphical user interface, and at the same time marks the position of the current setting value in the parameter value distribution chart. More content about the parameter value distribution chart may be found in operation 220 of FIG. 2.

In 1530, the target scanning protocol is generated in response to user adjustment instructions in the graphical user interface with respect to the current setting value.

The adjustment instruction may be used to adjust the current setting value, which is determined and input by the user based on the dashboard chart. The processing device may control a scanning device to scan a scanning object based on the target scanning protocol.

More descriptions about the target scanning protocol may be found in operation 230 in FIG. 2.

In this embodiment, based on the position of the current setting value annotated in the parameter value distribution chart, the user is informed of the health degree of the current setting value, and the user is able to adjust the current setting value of the protocol parameter based on the health degree to reduce or avoid wrong parameter settings in the target scanning protocol. In this way, a requirement for theoretical knowledges and practical experience of the user is reduced, which makes the manner for determining the scanning protocol more practical.

In some embodiments, the process 1500 further includes: updating the parameter value distribution chart based on the target scanning protocol.

After adjusting the current setting value of the target protocol parameter based on the adjustment instruction of the user, the processing device may obtain an adjusted current setting value. Based on the adjusted current setting value, the processing device may update the parameter value distribution chart corresponding to the scanning protocol, i.e., update the position of the current setting value annotated in the parameter value distribution chart. In this embodiment, by updating the parameter value distribution chart according to the generated target scanning protocol, a more accurate parameter value distribution chart is obtained, which facilitates subsequent use of the parameter value distribution chart.

FIG. 16 is a flowchart illustrating an exemplary process for updating a protocol database according to some embodiments of the present disclosure. In some embodiments, a process 1600 is performed by the processing device 120. As shown in FIG. 16, the process 1600 may include the following operations.

In 1610, a target scanning protocol or a combination of the target scanning protocol and a target image is sent to a cloud server for verification.

After obtaining the target scanning protocol, the processing device may send the target scanning protocol to the cloud server to verify the target scanning protocol, i.e., to determine whether a health degree of a current setting value in the target scanning protocol satisfies a preset requirement. The processing device may also send the target scanning protocol, along with the target image obtained by the scanning device via scanning the scanning object based on the target scanning protocol, to the cloud server.

An ecological database is stored in the cloud server. After the processing device sends the target scanning protocol or the target scanning protocol and the target image to the cloud server, the cloud server may store the received data in the ecological database. A manager obtains the target scanning protocol from the ecological database for verification according to a preset period. The manager is a specialized technician, e.g., a senior scanning device operator, etc.

In 1620, in response to that the cloud server completes the verification of the target scanning protocol, the protocol database is updated by adding the target scanning protocol to the protocol database.

In some embodiments, the protocol database is used to provide the parameter value distribution chart corresponding to the scanning protocol. The scanning protocols stored in the protocol database may be used as reference scanning protocols.

FIG. 17 is a schematic diagram illustrating a process for updating a protocol database according to some embodiments of the present disclosure.

As shown in FIG. 17, operations performed by a processing device may form a loop. After determining a target scanning protocol based on the obtained parameter value distribution chart, the processing device uploads the target scanning protocol to a cloud server, which includes an ecological database. In some embodiments, the cloud server includes a display device on which a target scanning protocol to be verified is displayed, and after a manager verifies the target scanning protocol based on a content displayed on the display device, a verification result is obtained. The verification result may include a verification passed and a verification failed. If the verification result is the verification passed, the manager may perform a confirmation operation on the display device, and the cloud server may respond to the confirmation operation of the manager by sending the target scanning protocol that passes the verification to a terminal device. If the verification fails, the manager may adjust a current setting value in the target scanning protocol and send the adjusted target scanning protocol (and the corresponding parameter value distribution chart) to the terminal device; after the terminal device receives the target scanning protocol or the adjusted target scanning protocol (i.e., in response to a completion of the verification of the target scanning protocol by the cloud server), the terminal device performs an update operation on the protocol database through the processing device, i.e., adds the target scanning protocol to the protocol database.

In this embodiment, the target scanning protocol or the target scanning protocol and the target image are sent to the cloud server for verification; in response to the completion of the verification on the target scanning protocol by the cloud server, the updating operation is performed on the protocol database. In this way, the protocol database is updated to improve an accuracy of the parameter value distribution chart in the protocol database, thereby making the scanning protocol determination manner more practical and reliable. Moreover, in this way, the protocol database is enriched and improved, and the terminal device is able to realize a cloud synchronization of the protocol database.

FIG. 18 is a flowchart illustrating an exemplary process for constructing a protocol database according to some embodiments of the present disclosure. In some embodiments, a process 1800 is performed by a processing device, such as the processing device 120. As shown in FIG. 18, the process 1800 includes the following operations.

In 1810, for each of different scanning protocols, scanning images collected by different setting values of protocol parameters of the scanning protocol are obtained.

Alternatively, the processing device may obtain a great number of historical scanning images of a great number of users and categorize the historical scanning images according to the scanning protocols to obtain the scanning images collected by different setting values of the protocol parameters of each scanning protocol.

In 1820, for each of the different scanning protocols, quality assessment results of the scanning images are obtained.

In some embodiments, after obtaining the above-described scanning images, the processing device obtains quality label information of the scanning images to determine the quality assessment result of each of the scanning images, or the processing device performs the quality assessment on the scanning images to determine the quality assessment result of each of the scanning images. Exemplarily, the processing device inputs each of the scanning images into an image quality assessment model separately to obtain the quality assessment results output by the image quality assessment model. More descriptions of the image quality assessment model may be found in related descriptions of FIG. 2.

In 1830, the protocol database is constructed based on the setting values of the protocol parameters corresponding to scanning images whose the quality assessment results satisfy quality requirements.

In some embodiments, for each scanning protocol, the processing device obtains scanning images whose the quality assessment results satisfy quality requirements, obtains a setting value of each protocol parameter corresponding to those scanning images, and further determines a reference value range of each protocol parameter according to the setting value of each protocol parameter. In this way, reference value ranges of each protocol parameter in each scanning protocol are determined, and these reference value ranges of all scanning protocols can form the protocol database.

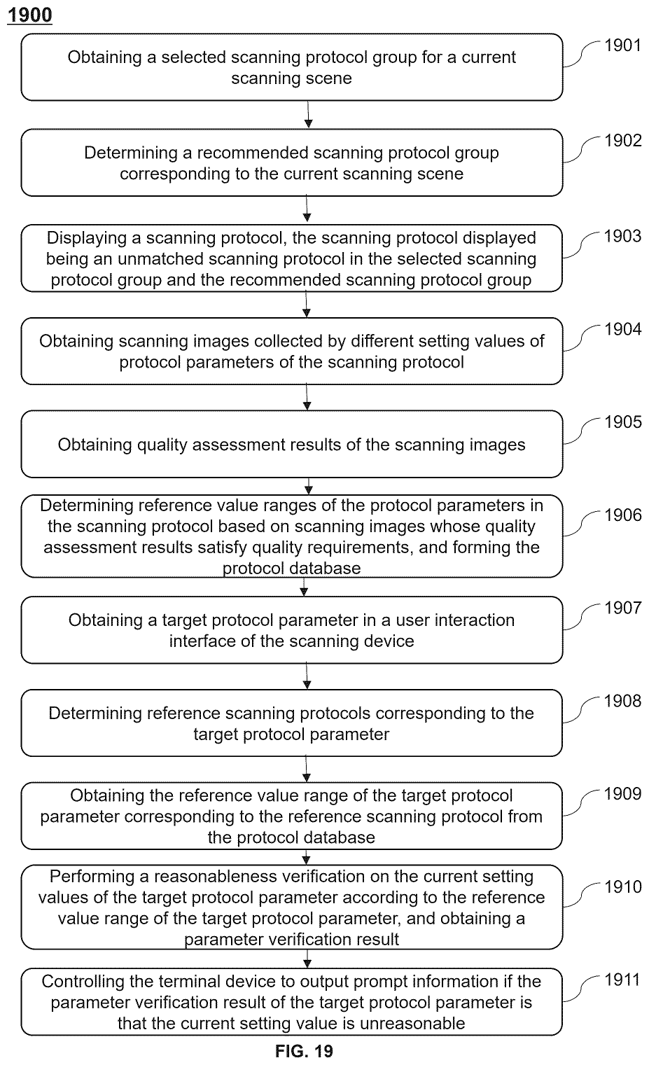

FIG. 19 is a flowchart illustrating an exemplary method for setting protocol parameters according to some embodiments of the present disclosure. In some embodiments, a process 1900 is performed by a processing device, such as the processing device 120. As shown in FIG. 19, the process 1900 includes the following operations.

In 1901, a selected scanning protocol group for a current scanning scene is obtained.

In 1902, a recommended scanning protocol group corresponding to the current scanning scene is determined. In some embodiments, at least one recommended scanning protocol is included within the recommended scanning protocol group.

In 1903, a scanning protocol is displayed, and the scanning protocol displayed being an unmatched scanning protocol in the selected scanning protocol group and the recommended scanning protocol group.

In 1904, scanning images collected by different setting values of protocol parameters of the scanning protocol are obtained.

In 1905, quality assessment results of the scanning images are obtained.

In 1906, reference value ranges of the protocol parameters in the scanning protocol are determined based on scanning images whose quality assessment results satisfy quality requirements, and the protocol database is formed.

In 1907, a target protocol parameter in a graphical user interface of the scanning device is obtained.

In 1908, a reference scanning protocol corresponding to the target protocol parameter is determined.

In 1909, the reference value range of the target protocol parameter corresponding to the reference scanning protocol is obtained from the protocol database.

In 1910, a reasonableness verification is performed on the current setting values of the target protocol parameter according to the reference value range of the target protocol parameter, and a parameter verification result is obtained.

In 1911, the terminal device is controlled to output prompt information if the parameter verification result of the target protocol parameter is that the current setting value is unreasonable.

More descriptions of the above operations 1901-1911 may be found in the relevant descriptions above (e.g., FIGS. 2-18), which are not repeated here.

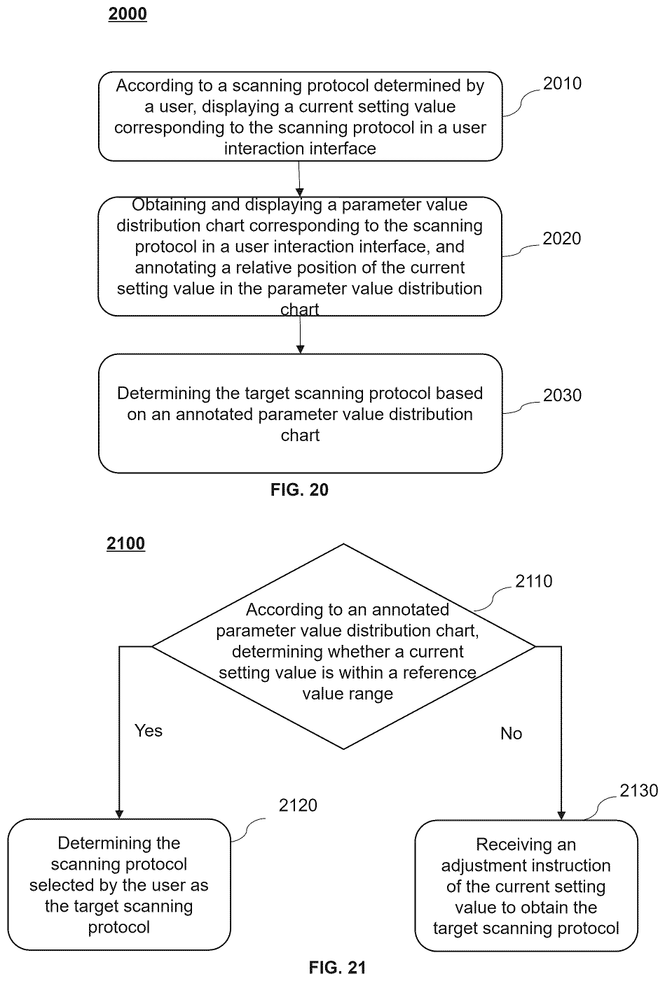

FIG. 20 is a flowchart illustrating an exemplary process for determining a target scanning protocol according to some embodiments of the present disclosure. In some embodiments, a process 2000 is performed by a processing device, such as the processing device 120. As shown in FIG. 20, the process 2000 includes the following operations.

In 2010, according to a scanning protocol determined by a user, a current setting value corresponding to the scanning protocol is displayed in a graphical user interface.

More descriptions about the scanning protocol and the current setting value may be found in operation 210 in FIG. 2.

In 2020, a parameter value distribution chart corresponding to the scanning protocol is obtained and displayed in the graphical user interface, and a relative position of the current setting value in the parameter value distribution chart is annotated.

More information about the parameter charter and the relative position may be found in operation 220 in FIG. 2.

In 2030, a target scanning protocol is determined based on an annotated parameter value distribution chart.

More descriptions about the target scanning protocol may be found in the descriptions of operation 230 in FIG. 2.

FIG. 21 is a flowchart illustrating an exemplary process for determining a target scanning protocol according to some embodiments of the present disclosure. In some embodiments, a process 2100 is performed by a processing device, such as the processing device 120. As shown in FIG. 21, the process 2100 includes the following operations.

In 2110, according to an annotated parameter value distribution chart, whether a current setting value is within a reference value range is determined.

The processing device may compare the current setting value and the reference value range based on the relative position of the current setting value in the parameter value distribution chart and determine whether the current setting value is within the reference value range. More descriptions of the reference value range may be found in FIG. 2.

In 2120, if the current setting value is within the reference value range, the scanning protocol selected by the user is determined as the target scanning protocol.

If the current setting value is within the reference value range, it indicates that the current setting value is frequently used by a great number of users, the scanning protocol corresponding to the current setting value is determined as the target scanning protocol. If the scanning protocol includes a plurality of protocol parameters, and the current setting value of each protocol parameter is within the corresponding reference value range, the scanning protocol is determined as the target scanning protocol.

In 2130, if the current setting value exceeds the reference value range, an adjustment instruction of the current setting value is received to obtain the target scanning protocol.

If the current setting value exceeds the reference value range, it indicates that the current setting value is not frequently used by the great number of other users.

When the processing device determines that the current setting value exceeds the reference value range, the user may adjust the current setting value according to the reference value range, i.e., adjust the current setting value to be within the reference value range. After receiving a modification on the current setting value by the user, the processing device determines the modified current setting value to be a target setting value of the target protocol parameter, and determines the scanning protocol including the target setting value of the target protocol parameter as the target scanning protocol.

In one embodiment, the method for setting protocol parameters further includes: displaying prompt information in the graphical user interface if the current setting value is abnormal. The prompt information may be a voice prompt, a text prompt, etc. For example, if the current setting value is determined to be abnormal, the processing device pops up a prompt box to display the abnormal protocol parameter, and the current setting value of the abnormal protocol parameter.

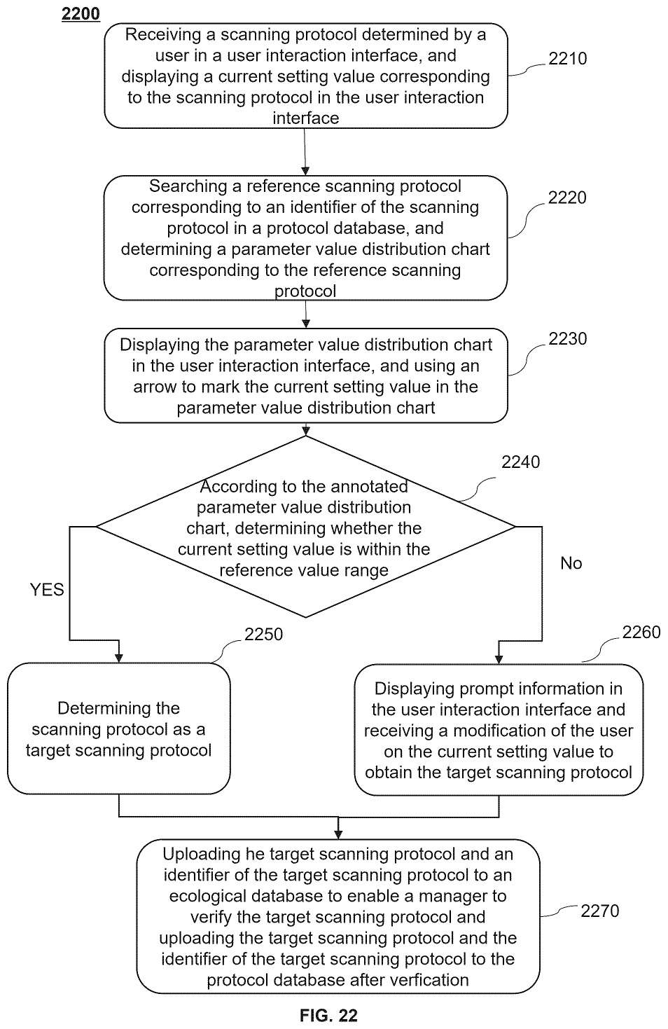

FIG. 22 is a flowchart illustrating an exemplary method for setting protocol parameters according to some embodiments of the present disclosure. In some embodiments, a process 2200 is performed by a processing device, such as the processing device 120. As shown in FIG. 22, the process 2200 includes the following operations.

In 2210, a scanning protocol determined by a user in a graphical user interface is received, and a current setting value corresponding to the scanning protocol is displayed in the graphical user interface. The scanning protocol determined by the user on a checklist interface may be referred to as a scanning protocol to be verified.

In 2220, a reference scanning protocol corresponding to an identifier of the scanning protocol is searched in a protocol database, and a parameter value distribution chart corresponding to the reference scanning protocol is determined.

In 2230, the parameter value distribution chart is displayed in the graphical user interface, and an arrow is used to mark the position of the current setting value in the parameter value distribution chart. The parameter value distribution chart may be used to indicate a statistical distribution situation of values of the protocol parameters corresponding to the scanning protocol.

In 2240, according to the annotated parameter value distribution chart, whether the current setting value is within the reference value range is determined.

In 2250, if the current setting value is within the reference value range, the scanning protocol is determined as a target scanning protocol.

In 2260, if the current setting value exceeds the reference value range, prompt information is displayed in the graphical user interface and a modification of the user on the current setting value is received to obtain the target scanning protocol.

In 2270, the target scanning protocol and an identifier of the target scanning protocol are uploaded to an ecological database to enable a manager to verify the target scanning protocol. After verification, the target scanning protocol and the identifier of the target scanning protocol are uploaded to the protocol database.

More description of the above operations 2010-2070 may be found in the relevant descriptions in the previous sections (e.g., FIGS. 2-18), which are not repeated here.

The basic concepts have been described above, and it is apparent to those skilled in the art that the foregoing detailed disclosure serves only as an example and does not constitute a limitation of the present disclosure. While not expressly stated herein, those skilled in the art may make various modifications, improvements, and amendments to the present disclosure. Those types of modifications, improvements, and amendments are suggested in the present disclosure, so those types of modifications, improvements, and amendments are still within the spirit and scope of the exemplary embodiments of the present disclosure.

Similarly, it should be noted that in order to simplify the presentation of the disclosure of the present disclosure, and thereby aiding in the understanding of one or more embodiments, the preceding description of embodiments of the present disclosure sometimes combines a variety of features into a single embodiment, accompanying drawings, or a description thereof. However, this manner of disclosure does not imply that the objects of the present disclosure require more features than those mentioned in the claims. Rather, claimed subject matter may lie in less than all features of a single foregoing disclosed embodiment.

For each of the patents, patent applications, patent application disclosures, and other materials cited in the present disclosure, such as articles, books, specification sheets, publications, documents, etc., are hereby incorporated by reference in their entirety into the present disclosure. Application history documents that are inconsistent with or conflict with the contents of the present disclosure are excluded, as are documents (currently or hereafter appended to the present disclosure) that limit the broadest scope of the claims of the present disclosure. It should be noted that in the event of any inconsistency or conflict between the descriptions, definitions, and/or use of terminology in the materials appended to the present disclosure and those described in the present disclosure, the descriptions, definitions, and/or use of terminology in the present disclosure shall prevail.

Finally, it should be understood that the embodiments described in the present disclosure are only used to illustrate the principles of the embodiments of the present disclosure. Other deformations may also fall within the scope of the present disclosure. As such, alternative configurations of embodiments of the present disclosure may be considered to be consistent with the teachings of the present disclosure as an example, not as a limitation. Correspondingly, the embodiments of the present disclosure are not limited to the embodiments expressly presented and described herein.

What is claimed is:

1. A method for setting protocol parameters, comprising:
obtaining a current setting value of a target protocol parameter in a scanning protocol to be verified, the current setting value being set by a user through a terminal device;
determining a statistical distribution situation of historical setting values of the target protocol parameter and a relative position of the current setting value in the statistical distribution situation; and
generating prompt information relating to parameter setting based on the relative position.

2. The method of claim 1, wherein the generating prompt information relating to parameter setting based on the relative position includes:
performing a reasonableness verification on the current setting value of the target protocol parameter based on the relative position to obtain a parameter verification result, the parameter verification result indicating whether the current setting value is reasonable; and
in response to determining that the parameter verification result indicates that the current setting value is unreasonable, generating the prompt information, the prompt information including a reference parameter value of the target protocol parameter.

3. The method of claim 1, wherein the determining a statistical distribution situation of historical setting values of the target protocol parameter includes:
determining target historical scanning protocols corresponding to the scanning protocol;
for each of the target historical scanning protocols, determining a quality assessment result of a scanning image corresponding to the target historical scanning protocol;
selecting reference scanning protocols whose quality assessment results satisfy preset requirements from the target historical scanning protocols; and
determining the statistical distribution situation based on the historical setting values of the target protocol parameter in the reference scanning protocols.

4. The method of claim 1, wherein the determining a statistical distribution situation of historical setting values of the target protocol parameter includes:
determining, based on a protocol database, a reference value range of the target protocol parameter as the statistical distribution situation, wherein the protocol database stores reference value ranges for a plurality of protocol parameters of different types of reference scanning protocols.

5. The method of claim 1, wherein before the user sets the current setting value via the terminal device, the method further includes:
obtaining a selected scanning protocol group for a current scanning scene, the selected scanning protocol group including at least one selected scanning protocol selected by the user;
determining a recommended scanning protocol group corresponding to the current scanning scene, the recommended scanning protocol group including at least one recommended scanning protocol; and
selecting the scanning protocol from the selected scanning protocol group and the recommended scanning protocol group and displaying the scanning protocol via the terminal device, the scanning protocol being included in the selected scanning protocol group but not included in the recommended scanning protocol group, or the scanning protocol being included in the recommended scan protocol group but not included in the selected scanning protocol group.

6. The method of claim 5, wherein the current scanning scene is determined by:
obtaining profile information of a scanning object, and determining the current scanning scene based on the profile information; or,
obtaining at least one protocol label based on the at least one selected scanning protocol included in the selected scanning protocol group, and determining the current scanning scene based on the at least one protocol label, wherein the at least one protocol label is used to identify a scanning body part.

7. The method of claim 1, wherein the statistical distribution situation includes a parameter value distribution chart,
the prompting information includes the parameter value distribution chart with an annotation, the annotation indicating a relative position of the current setting value in the parameter value distribution chart.

8. The method of claim 7, wherein the parameter value distribution chart includes one of a bar chart, a sector chart, a dashboard chart, and a rectangular chart;
different numerical bars in the bar chart indicate the frequency of use of different value ranges of the target protocol parameter;
different regions of the sector chart indicate the frequency of use of different value ranges of the target protocol parameter;
different regions of the dashboard chart indicate the frequency of use of different value ranges of the target protocol parameter, and the dashboard chart includes a pointer as the annotation; and
different regions in the rectangular graph indicate the frequency of use of different value ranges of the target protocol parameter.

9. The method of claim 7, wherein the parameter value distribution chart is determined by:
searching a reference scanning protocol corresponding to an identifier of the scanning protocol in a protocol database, and
determining the parameter value distribution chart of the protocol parameter based on a parameter value distribution chart corresponding to the reference scanning protocol, wherein the protocol database stores parameter value distribution charts of a plurality of protocol parameters of different types of reference scanning protocols.

10. The method of claim 9, further including:
generating a target scanning protocol based on the scanning protocol;
uploading the target scanning protocol and the identifier of the target scanning protocol to an ecological database for protocol verification; and
in response to determining that the target scanning protocol passes the protocol verification, saving the target scanning protocol and the identifier of the target scanning protocol into the protocol database.

11. The method of claim 7, further including:
determining, based on the parameter value distribution chart with the annotation, whether the current setting value of the target protocol parameter is within a reference value range; and
in response to determining that the current setting value of the target protocol parameter exceeds the reference value range, determining a reference parameter value according to the reference value range, wherein the prompt information further includes the reference parameter value.

12. The method of claim 7, wherein the scanning protocol includes a plurality of protocol parameters, parameter value distribution charts of the plurality of protocol parameters are displayed in a preset order, the preset order is determined based on an importance of the plurality of protocol parameters with respect to an imaging quality.

13. The method of claim 12, further including:
determining a scanning body part of a scanning object;
obtaining historical scanning images corresponding to the scanning body part and historical values of the protocol parameters corresponding to each historical scanning image;
determining an importance coefficient for each of the protocol parameters based on the historical scanning images and the historical values of the protocol parameters; and
determining the preset order based on the importance coefficient of each of the protocol parameters.

14. The method of claim 1, further including:
determining an associated protocol parameter that has an association with the target protocol parameter;
determining a parameter value of an association relationship parameter based on the current setting value of the target protocol parameter and a second current setting value of the associated protocol parameter; and
performing a reasonableness verification on the current setting value of the target protocol parameter and the second current setting value of the associated protocol parameter based on the parameter value of the association relationship parameter and a second statistical distribution situation corresponding to the association relationship parameter.

15. The method of claim 1, wherein the scanning protocol includes a plurality of protocol parameters, and the method further includes:
inputting current setting values of the protocol parameters in the scanning protocol into an image quality prediction model to obtain an image quality prediction result corresponding to the current setting values;
determining whether the image quality prediction result satisfies preset requirements; and
in response to determining that the image quality prediction result does not satisfy the preset requirements, receiving modified setting values of the protocol parameters input by the user through the terminal device until an image quality prediction result corresponding to the modified setting values satisfies the preset requirements.

16. The method of claim 2, wherein the reference parameter value is determined by:
obtaining profile information of a scanning object; and
determining the reference parameter value of the target protocol parameter by inputting the profile information and the statistical distribution situation of the historical setting values of the target protocol parameter into a parameter recommendation model, the parameter recommendation model being a trained machine learning model.

17. A system for setting protocol parameters, comprising a processing device and a display device, wherein:
the display device is configured to display a current setting value of a target protocol parameter on a graphical user interface,
the processing device is configured to obtain the current setting value, perform a reasonableness verification on the current setting value to generate a parameter verification result, and determine prompt information relating to parameter setting based on the parameter verification result, and
the display device is further configured to display the prompt information on the graphical user interface, wherein the prompt information indicates that the current setting value of the target protocol parameter needs to be adjusted.

18. The system of claim 17, further comprising a scanning device, wherein:
the display device is further configured to display a selected scanning protocol group on the graphical user interface before displaying the current setting value on the graphical user interface, the selected scanning protocol group including one or more selected scanning protocols;
the processing device is further configured to:
determine whether the selected scanning protocol group matches a current scanning scene;
in response to determining that the selected scanning protocol group does not match the current scanning scene, generate navigation information indicating suggestions for modifying the selected scanning protocol group; and
generate a target protocol group matching the current scanning scene in response to user adjustment operations on the graphical user interface; and
the scanning device is configured to execute the target protocol group under the control of the processing device to scan a scanning object.

19. A system for setting protocol parameters, comprising:
at least one storage device storing a set of instructions; and
at least one processor configured to communicate with the at least one storage device, wherein when executing the set of instructions, the at least one processor is configured to direct the system to perform operations including:
in response to user operations on a graphical user interface of a terminal device, selecting a scanning protocol and a current setting value of a target protocol parameter of the scanning protocol;
obtaining and displaying a parameter value distribution chart corresponding to the scanning protocol on the graphical user interface, the parameter value distribution chart having an annotation that indicates a relative position of the current setting value in the parameter value distribution chart; and
generating a target scanning protocol in response to user adjustment instructions on the graphical user interface with respect to the current setting value.

20. The system of claim 19, wherein the operations further comprise:
sending the target scanning protocol or a combination of the target scanning protocol and a target image to a cloud server for verification, wherein the target image is obtained via a scanning device by performing a scan on a scanning object according to the target scanning protocol, and the cloud server stores an ecosystem database; and
in response to that the cloud server completes the verification of the target scanning protocol, updating a protocol database by adding the target scanning protocol to the protocol database, wherein the protocol database is configured to store parameter value distribution charts corresponding to the scanning protocol.
